(19) United States
(12) Patent Application Publication
(10) Pub. No.: US 2024/0238436 A1
(43) Pub. Date: Jul. 18, 2024

(54) COMPOSITIONS INCLUDING CONJUGATED THERAPY ENHANCERS

(71) Applicant: BIOHAVEN THERAPEUTICS LTD., New Haven, CT (US)

(72) Inventors: Wieslaw KAZMIERSKI, Raleigh, NC (US); Richard PRACITTO, Wallingford, CT (US)

(21) Appl. No.: 18/555,855

(22) PCT Filed: May 17, 2022
(86) PCT No.: PCT/US2022/029533

§ 371 Date: Oct. 17, 2023

Related U.S. Application Data
(60) Provisional application No. 63/189,503, filed on May 17, 2021.

(51) Int. Cl. A61K 47/68; A61K 45/06; A61K 47/64; C07K 7/06; C07K 16/18

(52) U.S. Cl. CPC A61K 47/6811; A61K 45/06; A61K 47/64; A61K 47/6843; A61K 47/6889; C07K 7/06; C07K 16/18

(57) ABSTRACT

A composition including a first compound having the structure P-N-L^{PM}-MOI, wherein P-N is a protein agent moiety comprising a lysine residue, L^{PM} is a linker, and MOI is a moiety of interest, and a second compound having the structure LG-OH, wherein LG is a group comprising a target binding moiety that binds to a target agent.

### CROSS REFERENCE TO RELATED APPLICATION

This application claims priority to application No. 63/189,503, filed May 17, 2021, which is hereby incorporated by reference in its entirety.

### FIELD OF THE DISCLOSURE

The present disclosure relates to conjugated therapy enhancers that are useful for preventing and/or treating various conditions, disorders, or diseases. Specifically, the present disclosure relates to protein conjugates such as antibody-drug conjugates that are capable of acting as therapy enhancers.

### BACKGROUND

Conjugated therapy enhancers have been extensively used for preventing and/or treating various conditions, disorders, and diseases. Such enhancers typically include a therapeutically active molecule, such as an antibody, linked to a moiety having affinity to a particular target implicated in the condition, disorder, or disease. However, the majority of known conjugation techniques are not directed to a specific site of the therapeutically active molecule, and usually result in a mixture of conjugates. There remains a need in the development of site-specific conjugation techniques that provide reaction products with high degree of homogeneity.

### SUMMARY

The present disclosure is directed to compositions that include therapy enhancer agents containing moieties of interest conjugated to target agent moieties at specific locations.

In an embodiment, provided is a composition including;
a first compound having the structure of formula (P-II):

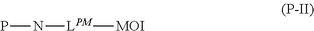

wherein:
P-N is a protein agent moiety including a lysine residue;
L^{PM} is a linker; and
MOI is a moiety of interest; and
a second compound having the structure:

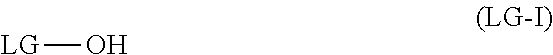

wherein LG is a group including a target binding moiety that binds to a target agent.

In another embodiment, the composition further includes:
A third compound having the formula (R-I):

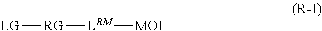

LG is a group including a target binding moiety that binds to a target agent, which is identical to LG in formula (LG-I);
RG is a reactive group;
L^{RM} is a linker, which is identical to in formula (P-II); and MOI is a moiety of interest.
A fourth compound having the formula (R-III):

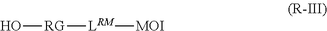

or a combination thereof.

### BRIEF DESCRIPTION OF THE DRAWINGS

FIG. 1. A representative UPLC/UV280 nm trace is provided for compound I-36 (MATE reagent I-20 conjugated to IVIG). The UPLC conditions are given in Example 2. uABT indicates universal Antibody Binding Terminal, DAR is Drug Antibody Ratio.

FIG. 2. UPLC traces of unconjugated IVIG (FIG. 2A), MATE impurities pooled standard (FIG. 2B), and I-36 Conjugate (FIG. 2C).

FIG. 3. Calibration curves for uABT, MATE-Linker, and MATE-Reagent pooled and individual impurities. Samples are prepared and analyzed by the methods given in Example 2.

### DETAILED DESCRIPTION

The following detailed description is provided to aid those skilled in the art in practicing the present invention. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its.

The articles “a” and “an” refer to one or to more than one (i.e., to at least one) of the grammatical object of the article unless the context clearly indicates otherwise. By way of example, “an element” means one element or more than one element.

As used herein, when specific definition is not otherwise provided, the term “substituted” refers to a group substituted with deuterium, a halogen (—F, —Cl, —Br, —I), a hydroxy group (—OH), an amino group (—NH₂), a carboxyl group (—CO₂H), a substituted or unsubstituted C1-C10 amine group, a nitro group (—NO₂), a C1-C10 alkyl group, a C3-C10 cycloalkyl group, a C6-C12 aryl group, a C1-C10 alkoxy group, a C1 to C10 trifluoroalkyl group such as a trifluoromethyl group (—CF₃) and the like, or a cyano group (—CN) instead of at least one hydrogen of a substituting group or compound.

Additional aspects will be set forth in part in the description which follows and, in part, will be apparent from the description.

The starting materials useful for making the pharmaceutical compositions of the present disclosure are readily commercially available or can be prepared by those skilled in the art.

The present disclosure is directed to compositions that include therapy enhancer agents containing moieties of interest conjugated to target agent moieties at specific locations.

In an embodiment, provided is a composition including;
A first compound having the structure of formula (P-II):

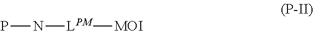

wherein:
P-N is a protein agent moiety including a lysine residue;
L^{PM} is a linker; and
MOI is a moiety of interest; and
A second compound having the structure:

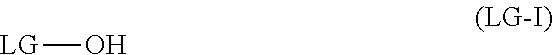

wherein LG is a group including a target binding moiety that binds to a target agent.

In another embodiment, the composition further includes:
a third compound having the formula (R-I):

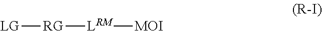

LG is a group including a target binding moiety that binds to a target agent, which is identical to LG in formula (LG-I);
RG is a reactive group;
L^{RM} is a linker, which is identical to in formula (P-II); and
MOI is a moiety of interest.
A fourth compound having the formula (R-III):

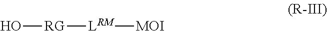

or a combination thereof.

The above compounds having the structure of formulae (P-II), (LG-I), (R-I), and (R-III) are described in detail in International Application No. PCT/US20/61127 filed Nov. 18, 2020, which is incorporated herein in its entirety by reference.

### Targets

Those skilled in the art after reading the present disclosure will appreciate that provided technologies herein are useful for conjugating various target agents to many types of moieties of interest. In some embodiments, provided technologies are particularly useful for conjugating protein agents with various moieties of interest. In some embodiments, target agents are or include a protein agent, a nucleic acid, or a combination thereof.

In some embodiments, a target agent is or includes a protein agent. In some embodiments, a target agent is a protein agent. In some embodiments, a target agent is a natural protein in a cell, tissue, organ or organism. In some embodiments, a target agent is an endogenous protein. In some embodiments, a target agent is an exogenous protein. In some embodiments, a target agent is a manufactured protein, e.g., a protein produced using various biotechnologies. In some embodiments, a target agent is an antibody agent. In some embodiments, a target agent is an antibody useful as therapeutics. Various such antibodies are known in the art and can be utilized as target agents. In some embodiments, an antibody is a monoclonal antibody. In some embodiments, an antibody is a polyclonal antibody. In some embodiments, an antibody is an lgG antibody. In some embodiments, an antibody is IVIG (in some embodiments, pooled from healthy donors). In some embodiments, a protein includes a Fc region. In some embodiments, an antibody includes a Fc region. In some embodiments, a Fc region includes a single heavy chain or a fragment thereof. In some embodiments, a Fc region includes two heavy chains or fragments thereof. In some embodiments, an antibody is a human antibody. In some embodiments, an antibody is a chimeric antibody. In some embodiments, an antibody is a humanized antibody. In some embodiments, an antibody is a mouse antibody.

In some embodiments, when characterizing polyclonal antibody agents or IVIG agents, either before, during or after conjugation, digestions are performed, e.g., enzyme digestions using IdeZ, IdeS, etc., so that certain regions of antibodies (e.g., Fab) are removed to provide compositions with improved homogeneity for characterization (e.g., by MS).

In some embodiments, an antibody is a therapeutic antibody, e.g., a FDA-approved antibody for therapeutic uses. In some embodiments, a therapeutic antibody is useful for treating cancer. In some embodiments, an antibody is adalimumab, alemtuzumab, atezolizumab, avelumab, ipilimumab, cetuximab, daratumumab, dinutuximab, elotuzumab, ibritumomab tiuxetan, imgatuzumab, infliximab, ipilimumab, necitumumab, obinutuzumab, ofatumumab, pertuzumab, reslizumab, rituximab, trastuzumab, mogamulizumab, AMP-224, FS-102, GSK-2857916, ARGX-111, ARGX-110, AFM-13, APN-301, BI-836826, BI-836858, enoblituzumab, otlertuzumab, veltuzumab, KHK-4083, BIW-8962, ALT-803, carotuximab, epratuzumab, inebilizumab, isatuximab, margetuximab, MOR-208, ocaratuzumab, talacotuzumab, tremelimumab, benralizumab, lumiliximab, MOR-208, Ifibatuzumab, GSK2831781, SEA-CD40, KHK-2823, or BI836858. In some embodiments, an antibody is rituximab, basiliximab, infliximab, cetuximab, siltuximab, dinutuximab, altertoxaximab, daclizumab, palivizumab, trastuzumab, alemtuzumab, omalizumab, efalizumab, bevacizumab, natalizumab, tocilizumab, eculizumab, mogamulizumab, pertuzumab, obinutuzumab, vedolizumab, pembrolizumab, mepolizumab, elotuzumab, daratumumab, ixekizumab, reslizumab, and atezolizumab, adalimumab, panitumumab, golimumab, ustekinumab, canakinumab, ofatumumab, denosumab, ipilimumab, belimumab, raxibacumab, ramucirumab, nivolumab, secukinumab, evolocumab, alirocumab, necitumumab, brodalumab, or olaratumab. In some embodiments, an antibody is daratumumab. In some embodiments, an antibody is cetuximab. In some embodiments, a provided compound or agent including an antibody agent moiety is useful for treating a condition, disorder or disease that may be treated by the antibody agent.

Antibodies may be prepared in a number of technologies in accordance with the present disclosure. In some embodiments, antibodies may have engineered structures compared to natural immunoglobulins. In some embodiments, antibodies may include certain tags for purification, identification, assessment, etc. In some embodiments, antibodies may contain fragments (e.g., CDR and/or Fc, etc.) and not full immunoglobulins. Those skilled in the art appreciate that when a site of an antibody is recited in the present disclosure (e.g., K246, K248, K288, K290, K317, etc .; unless indicated otherwise, human antibody per EU numbering), an amino acid residue may not be at the exact numbered site but may be at a site that corresponds to that numbered site per, e.g., EU numbering and/or sequence homology (e.g., homologues of the same or different species).

As those skilled in the art will appreciate, provided technologies among other things can provide directed conjugation with native targets, e.g., native antibodies. In some embodiments, target agents are or include native antibody agents. In some embodiments, target agents are or include engineered antibody agents. In some embodiments, target agents, e.g., antibodies, include no engineered unnatural amino acid residues.

### Target Binding Moieties

In some embodiments of formulae (LG-I) and (R-I):
LG is R^{LG}-L^{LG};
R^{LG} is

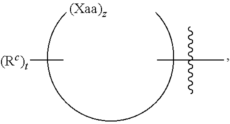

R^{c}-(Xaa)z-, a nucleic acid moiety, or a small molecule moiety;
each Xaa is independently a residue of an amino acid or an amino acid analog;
t is 0-50;
z is 1-50;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, S—(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, -L^{LG1}L^{LG2}, -L^{LG1}-L^{LG2}-L^{LG3}-, or -L^{LG1}-L^{LG2}-L^{LG3}-L^{LG4}-;
RG is -L^{RG1}-L^{RG2}-, -L^{LG4}-L^{RG1}-L^{RG2}-, -L^{LG3}-L^{LG4}-L^{RG1}-L^{RG2}-, or -L^{LG2}-L^{LG3}-L^{LG4}-L^{RG1}-L^{RG2}-;
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group including one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, —Cy—, —C(R′)₂—, —O—, —S—, —S—S—, —N(R′)—, —C(O)—, C—(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, P—(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiment, LG is or includes a target binding moiety that binds to a target agent, wherein the target agent is an antibody agent.

In some embodiments, LG is or includes a target binding moiety that binds to a Fc region, and/or R^{LG} is or includes DCAWXLGELVWCT (SEQ ID NO:1), wherein the two cysteine residues optionally form a disulfide bond, and X is an amino acid residue.

In some embodiments, LG is or includes a target binding moiety having the structure of A-1 to A-50:

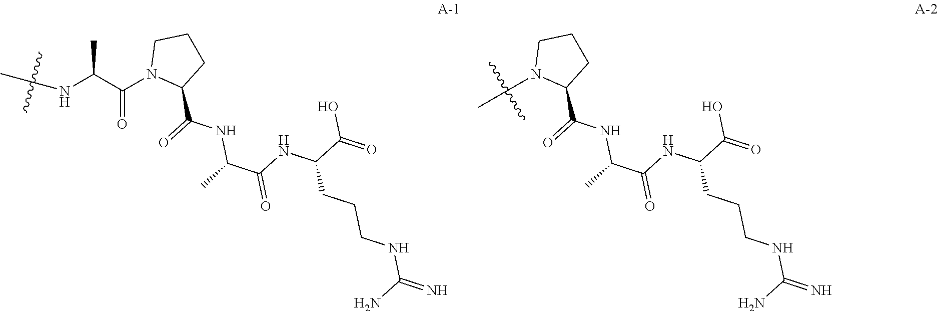 [SMILES: *N[C@@H](C)C(=O)N1CCC[C@H]1C(=O)N[C@@H](C)C(=O)N[C@@H](CCCNC(=N)N)C(=O)O ; *N1CCC[C@H]1C(=O)N[C@@H](C)C(=O)N[C@@H](CCCNC(=N)N)C(=O)O]

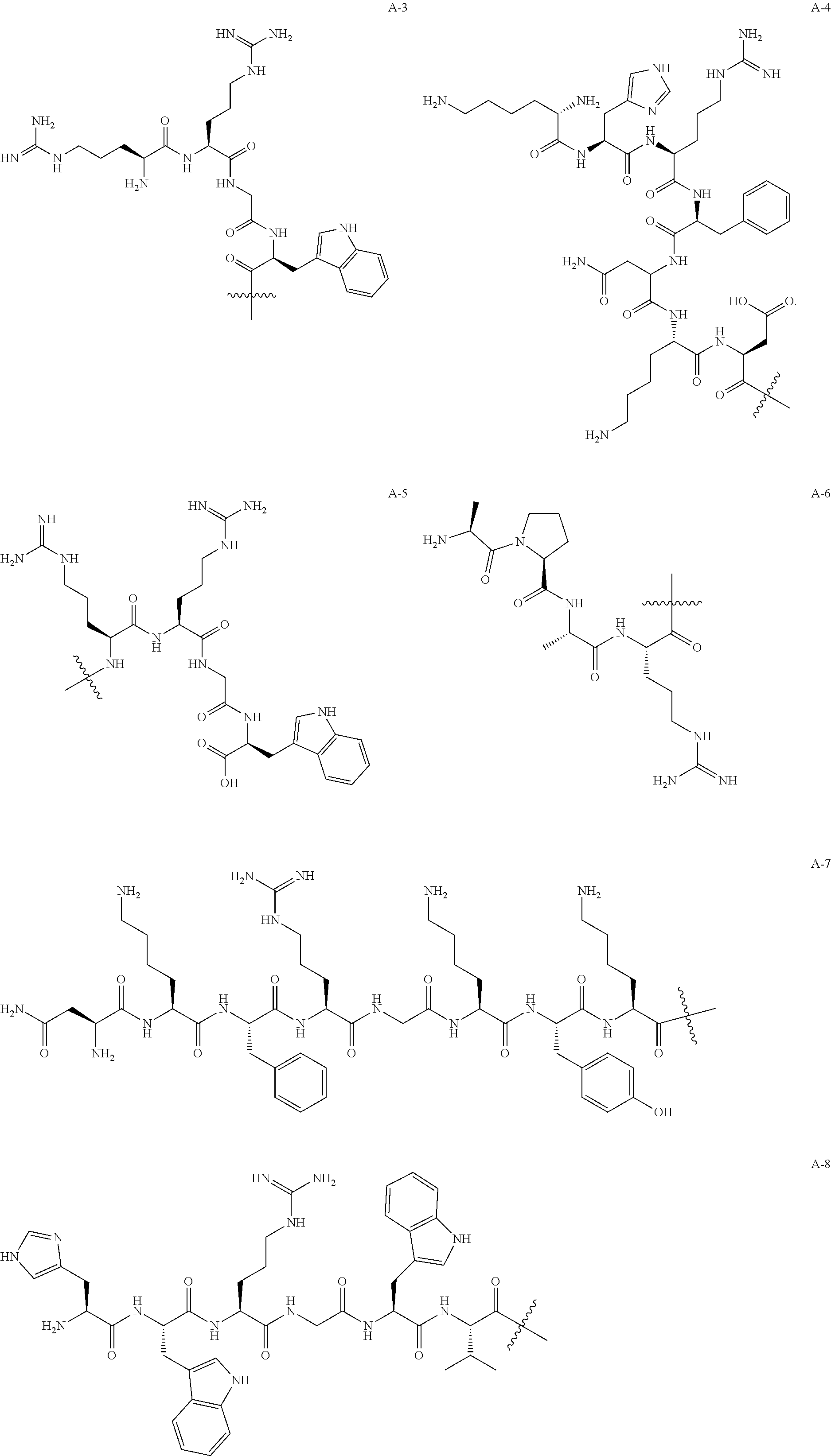

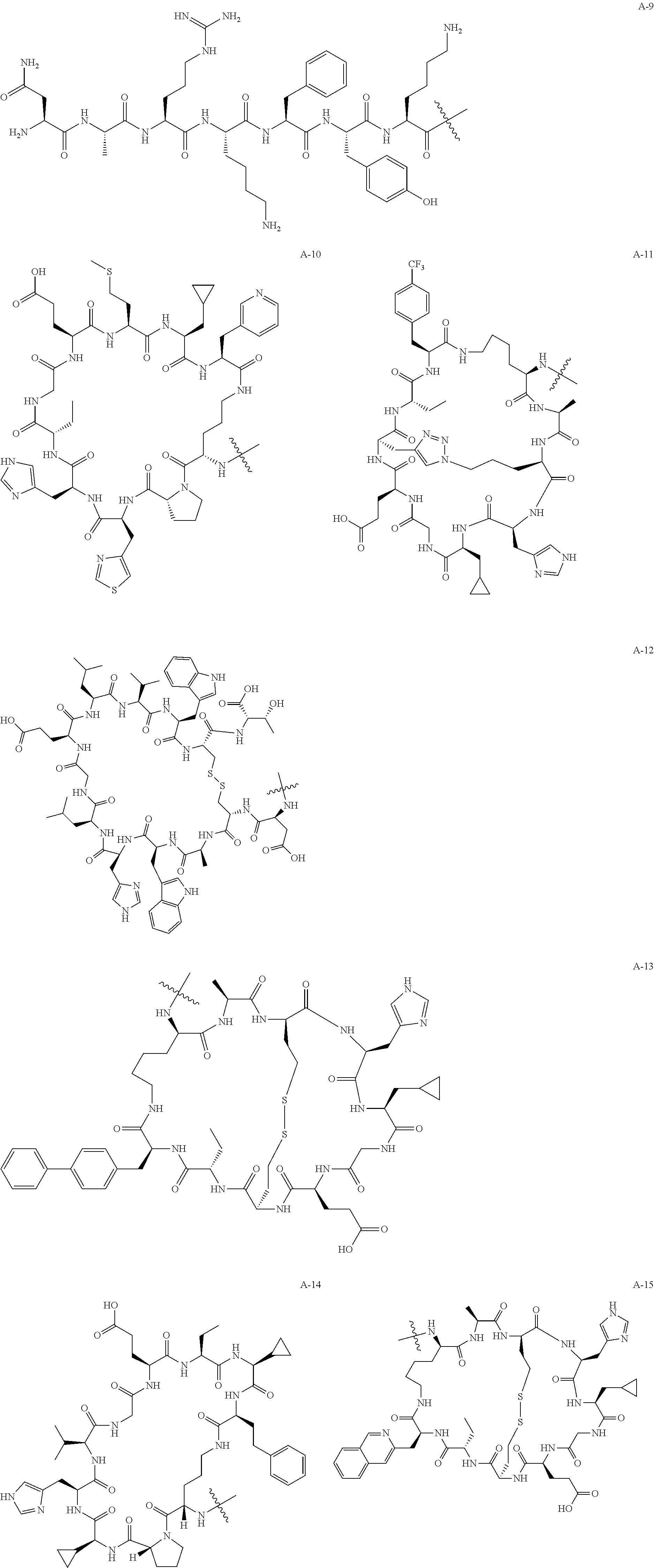

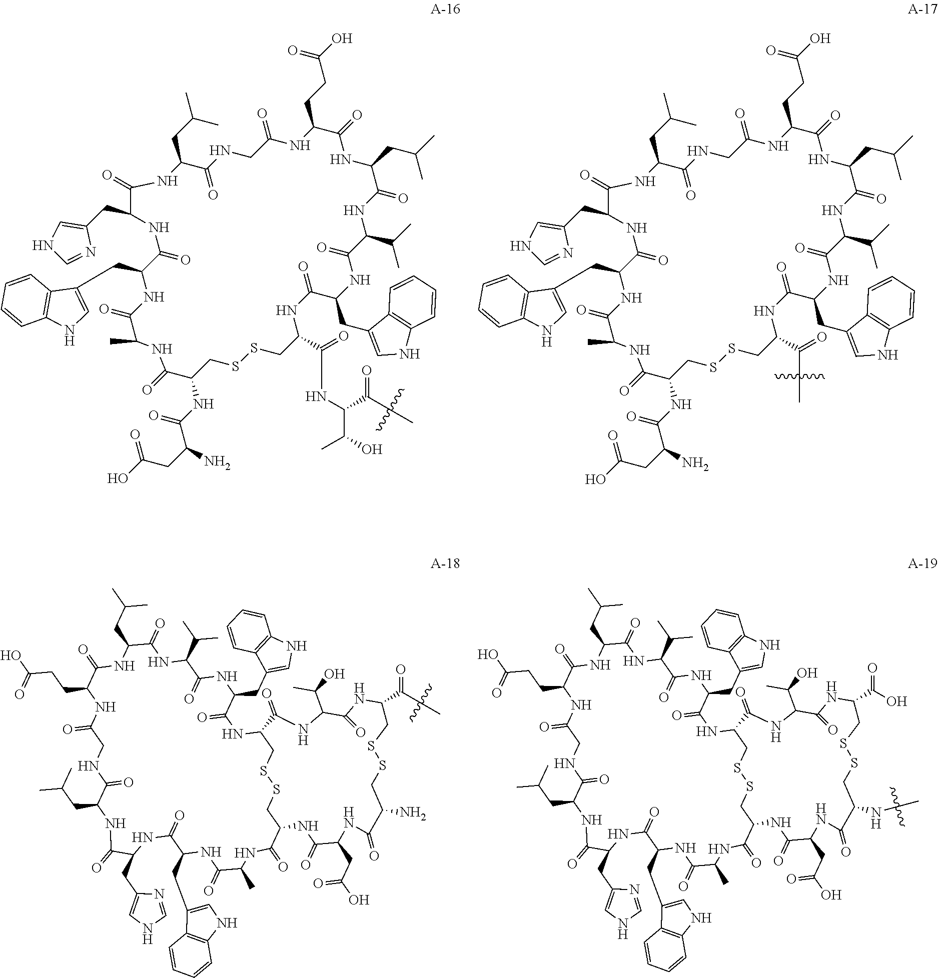

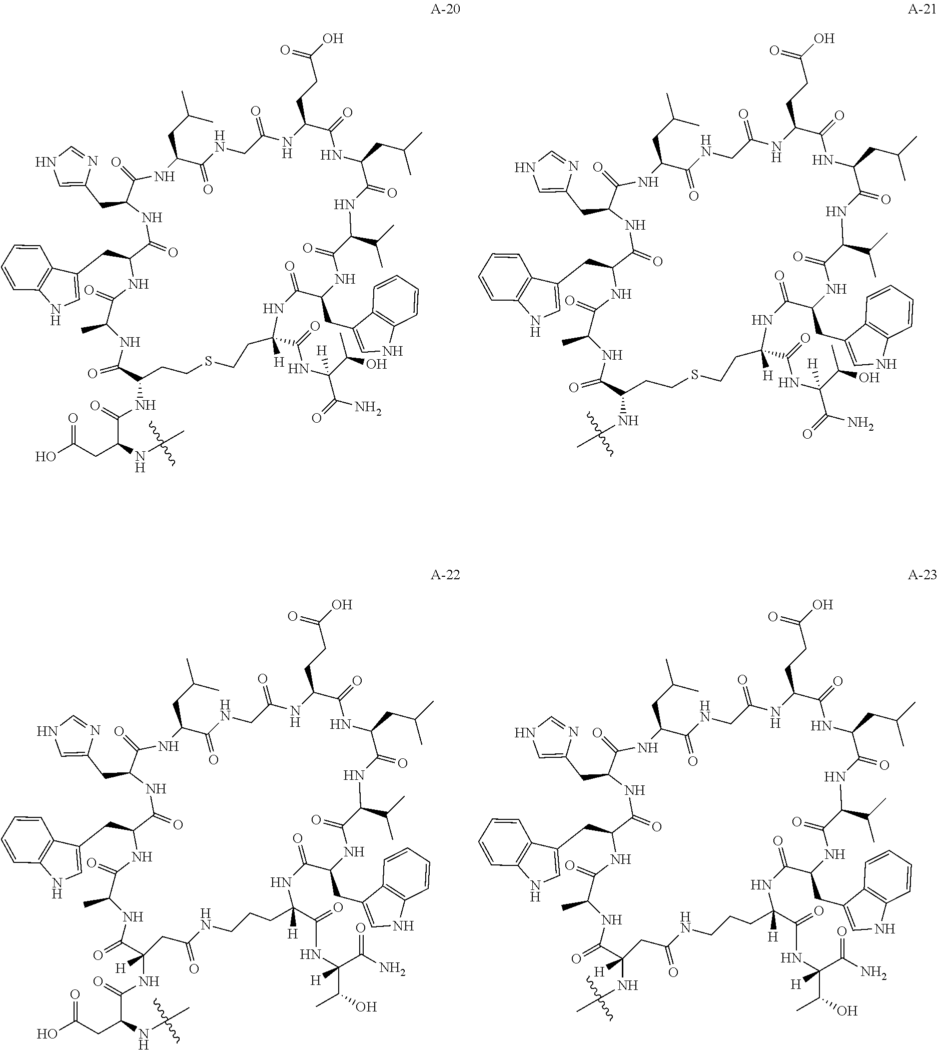

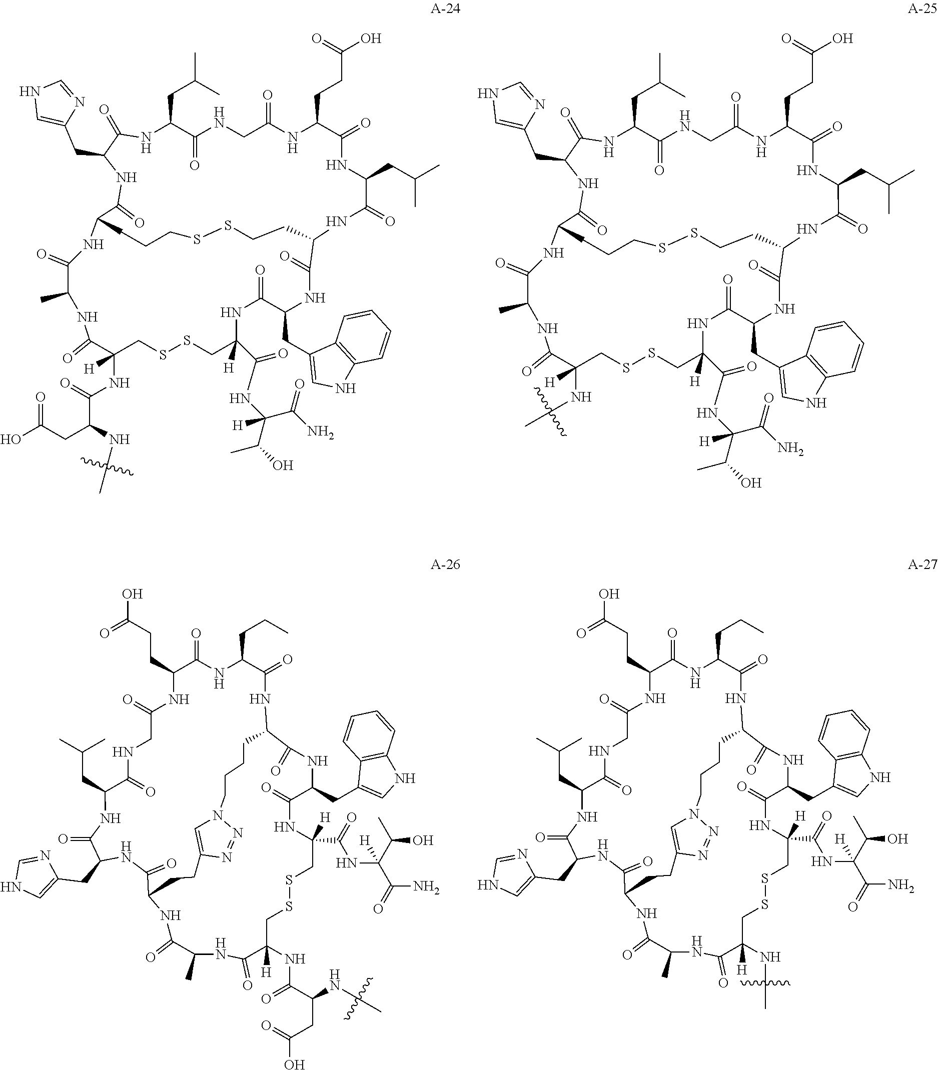

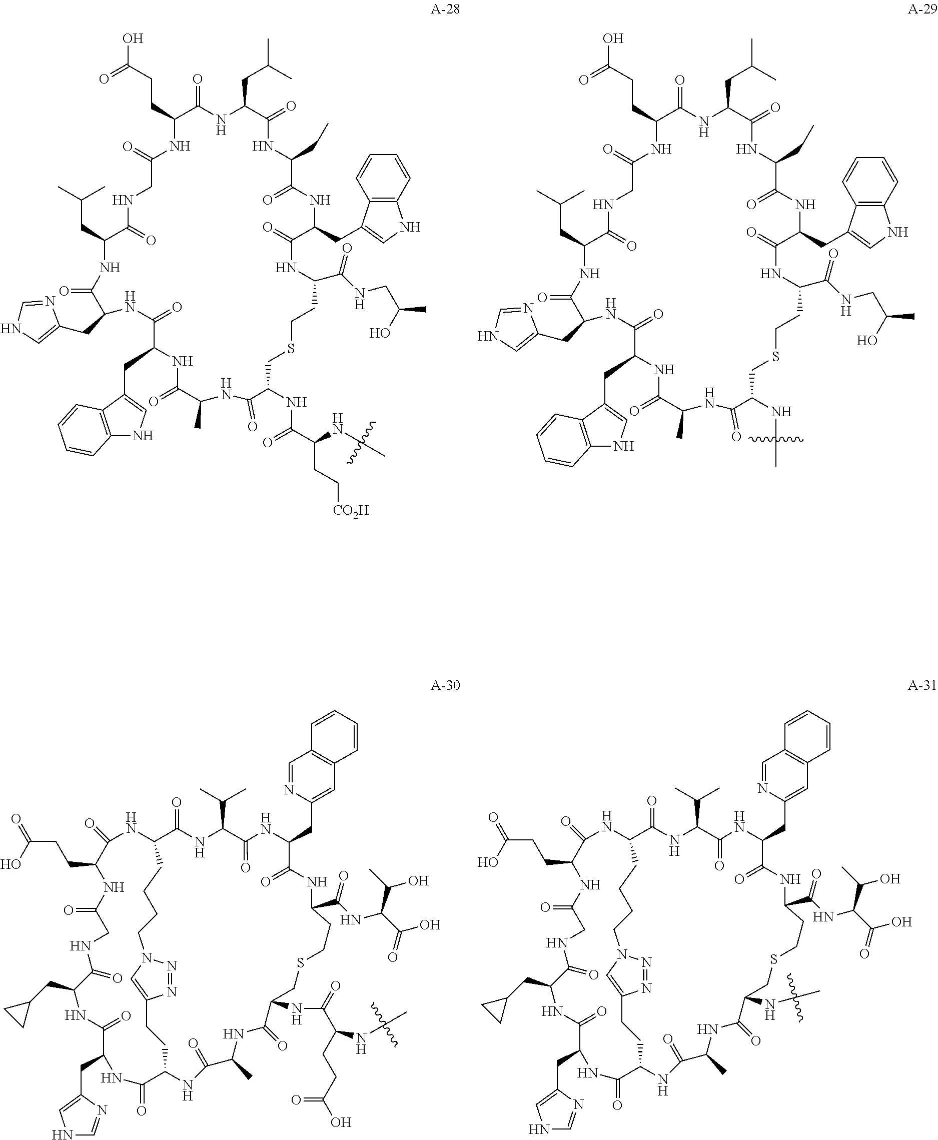

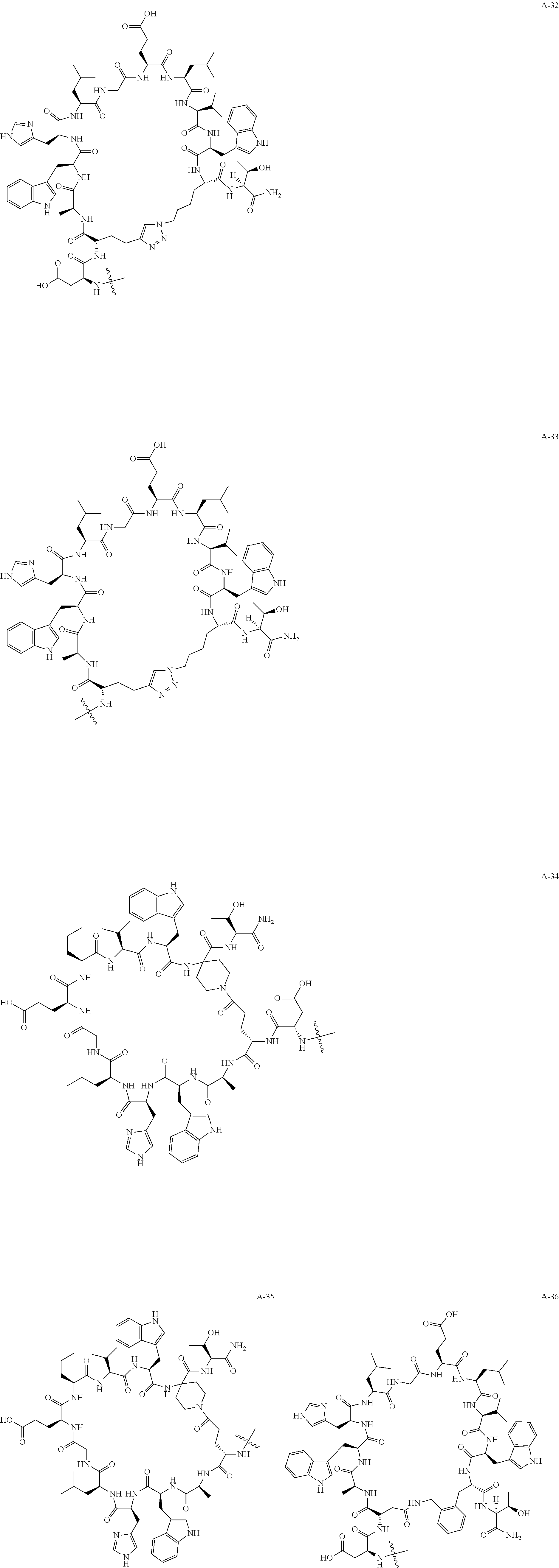

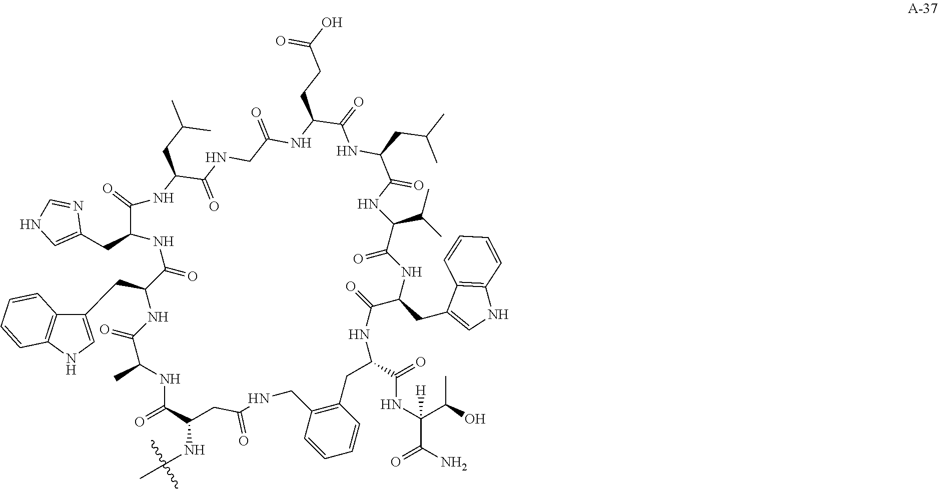

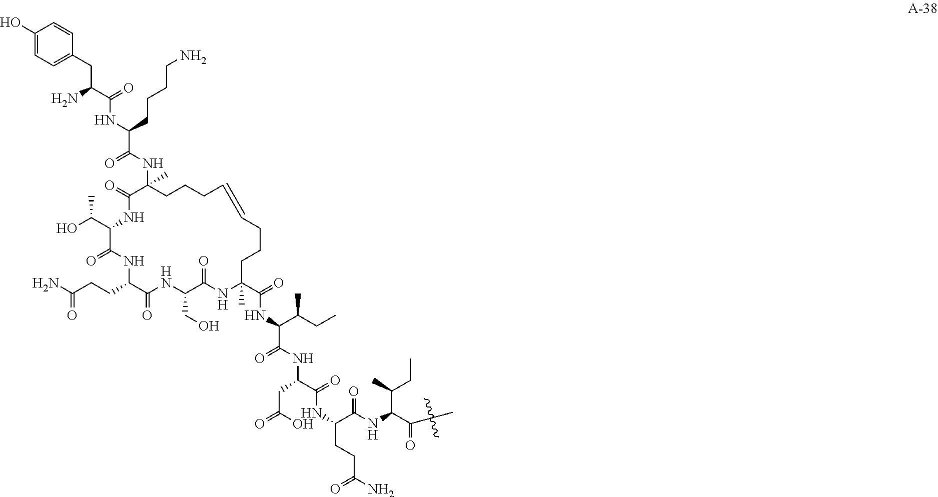 [SMILES: *C(=O)[C@@H](NC(=O)[C@H](CCC(N)=O)NC(=O)[C@H](CC(=O)O)NC(=O)[C@@H](NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1)[C@@H](C)CC)[C@@H](C)CC]

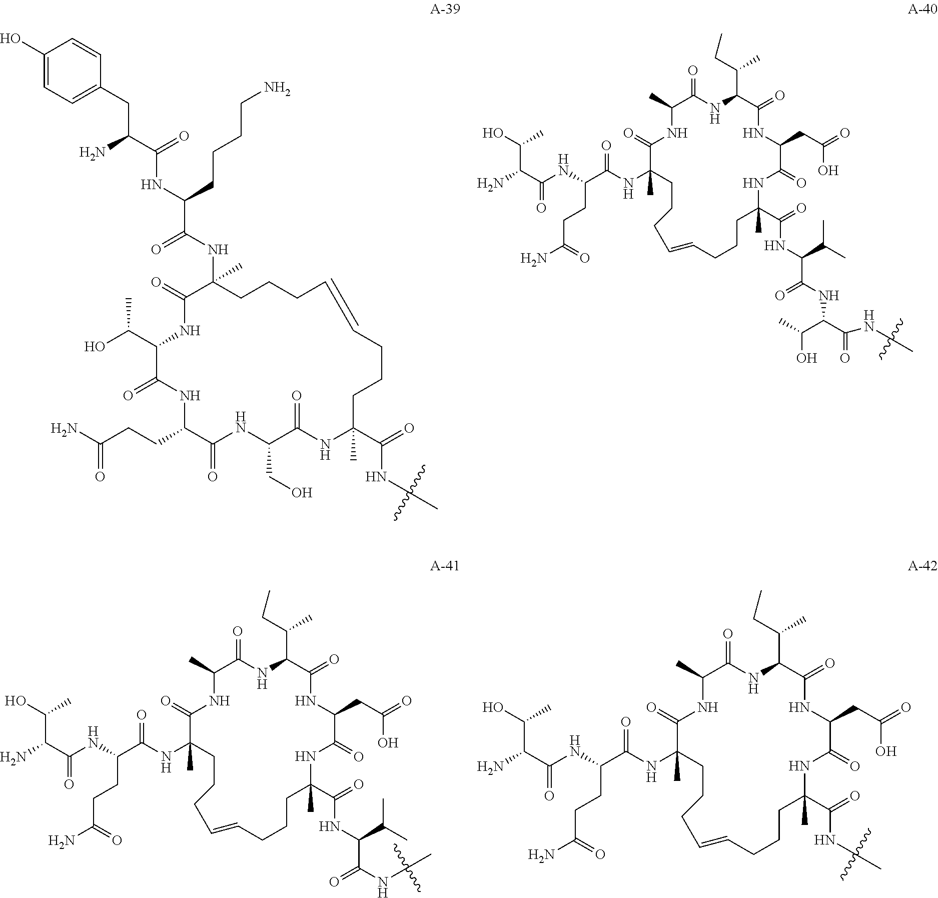

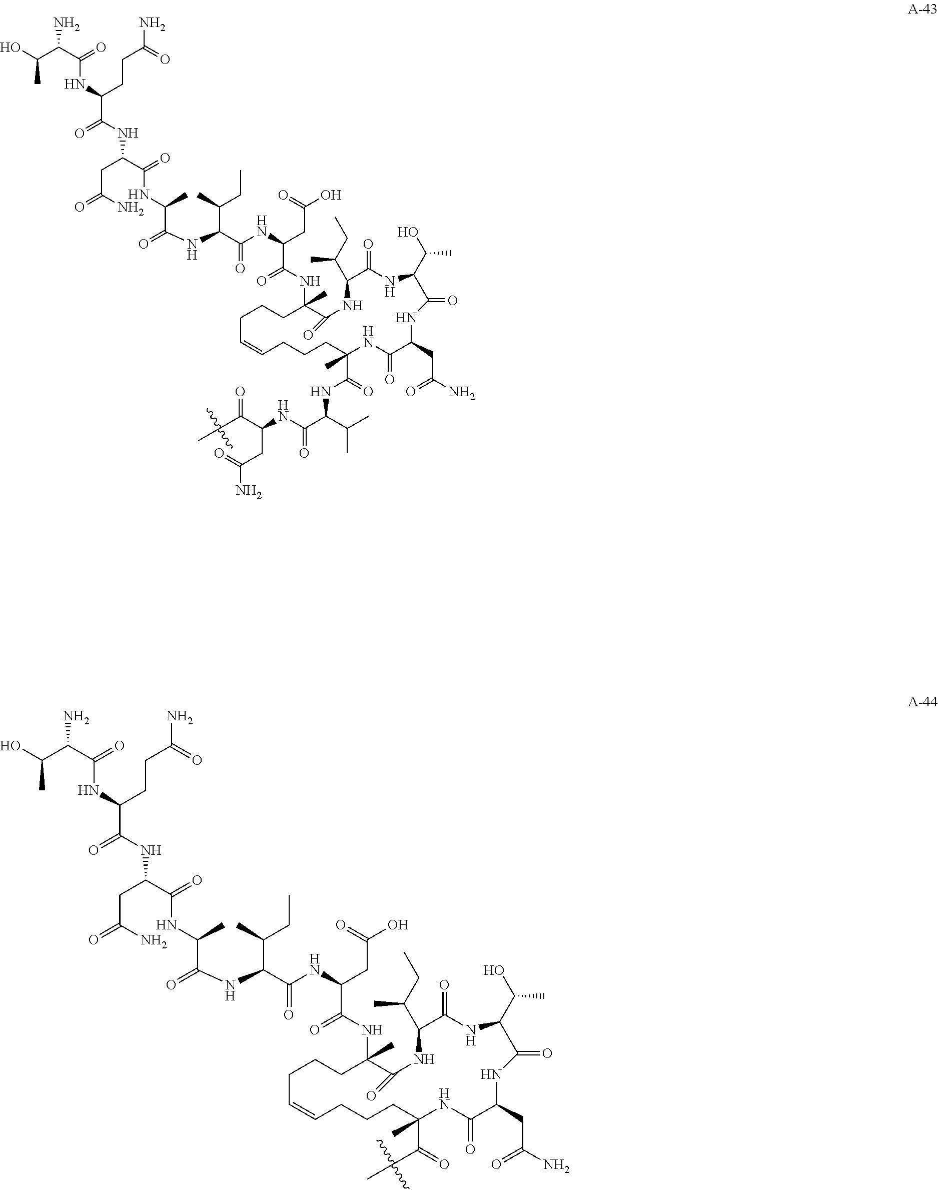

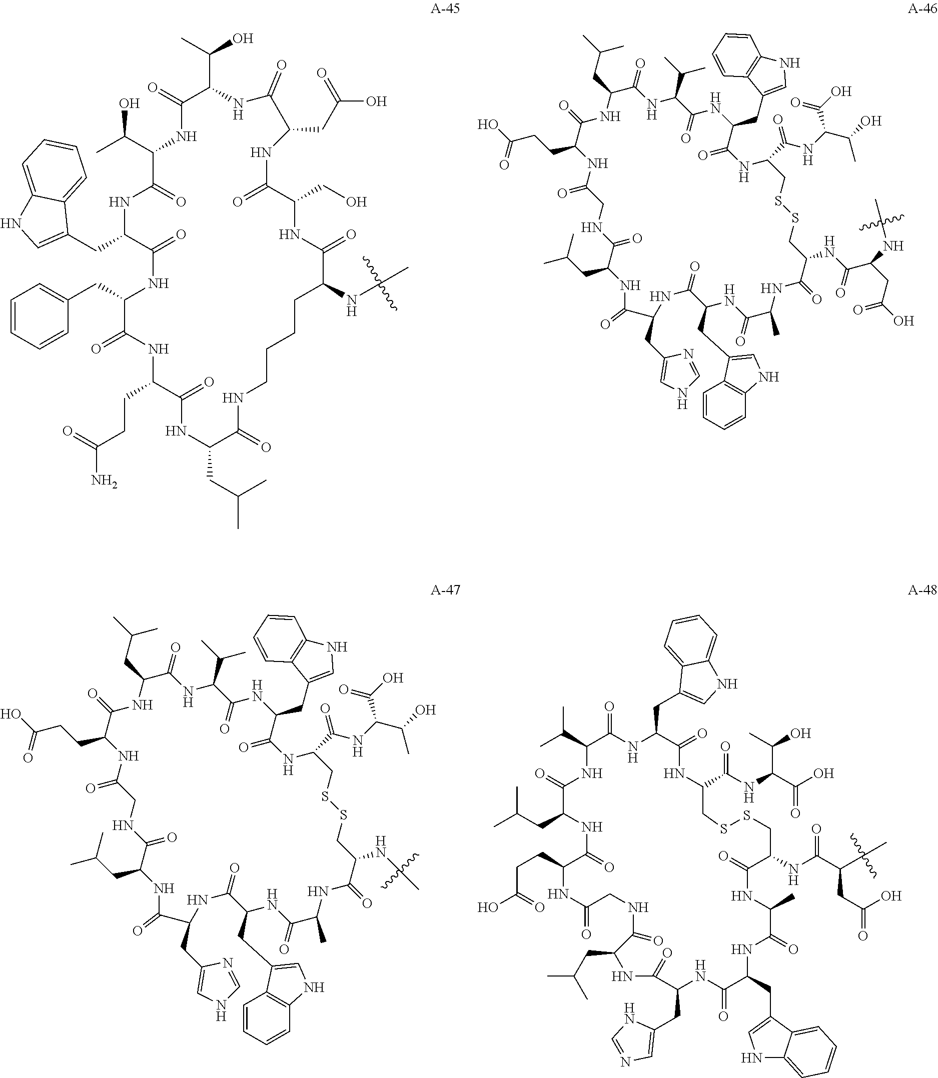

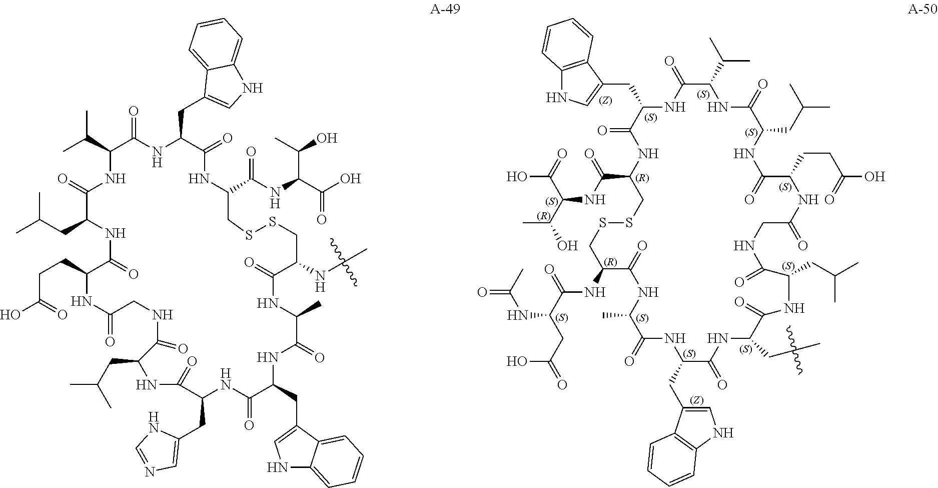

### Moieties of Interest

Those skilled in the art reading the present disclosure will appreciate that various types of moieties of interest can be utilized for various purposes in accordance with the present disclosure.

In some embodiments, moieties of interest are or include detectable moieties. Among other things, such moieties can be useful for detection, quantification, diagnosis, treatment, etc. In some embodiments, a moiety of interest is or includes a radioactive label. In some embodiments, a moiety of interest is or includes a label that can be detected through spectroscopy. In some embodiments, a moiety of interest is or includes a fluorophore such as FITC moiety.

A moiety of interest may be a moiety having affinity to a particular target implicated in a medical condition, disorder, or disease. In some embodiments, moieties of interest are or include therapeutic agent moieties. In some embodiments, a moiety of interest is or includes a drug moiety, e.g., a drug moiety in an antibody-drug conjugate. In some embodiments, a moiety of interest is or includes a toxic agent. In some embodiments, a moiety of interest is or includes a cytotoxic agent. In some embodiments, a moiety of interest is or includes an anti-cancer agent. In some embodiments, an anti-cancer agent is a chemotherapeutic agent.

In some embodiments, moieties of interest are or include moieties that can interact and/or recruit other agents, such as proteins, nucleic acids, cells, etc. In some embodiments, moieties of interest interact with proteins expressed by certain cell types, e.g., immune cells, disease cells, etc. In some embodiments, moieties of interest are immune cell binders. In some embodiments, moieties of interest recruit immune cells. In some embodiments, moieties of interest trigger, promote and/or enhance one or more immune activities, e.g., for removing, killing, and/or inhibiting desired targets (e.g., cancer cells, antigens, etc.). In some embodiments, moieties of interest interact, recruit and/or bind to disease cells, and trigger, promote and/or enhance removing, killing, and/or inhibiting disease cells.

In some embodiments, a moiety of interest is or includes a small molecule agent (e.g., one can bind specifically to its protein targets, cells targets, etc.). In some embodiments, a moiety of interest is or includes a peptide or protein agent (e.g., scFv, a peptide binder to specific target, etc.). In some embodiments, a moiety of interest is or includes a nucleic acid agent (e.g., an oligonucleotide, mRNA, etc.). In some embodiments, a moiety of interest is or includes a carbohydrate agent. In some embodiments, a moiety of interest is or includes a lipid agent.

In some embodiments, a moiety of interest is or includes a protein complex (e.g., Fab). In some embodiments, a moiety of interest is or includes a fluorophore. In some embodiments, a moiety of interest is or includes a cytotoxic small molecule agent. In some embodiments, a moiety of interest is or includes a cytotoxic peptide agent.

In some embodiments, a moiety of interest is an adjuvant. Those skilled in the art will appreciate various adjuvants can be utilized as moieties of interest in accordance with the present disclosure. In some embodiments, an adjuvant is one described in US 2019/0015516, which is incorporated herein in its entirety by reference. In some embodiments, a moiety of interest stimulates an immune system.

In some embodiments, a moiety of interest is or includes a particle. In some embodiments, a particle is or includes a nanoparticle.

In some embodiments, a moiety of interest is or includes a nucleic acid moiety. In some embodiments, a moiety of interest is or includes an oligonucleotide. In some embodiments, a moiety of interest is or includes an aptamer.

In some embodiments, a moiety of interest is an antibody agent. In some embodiments, a moiety of interest is or includes an antibody fragment. In some embodiments, a moiety of interest is an antibody agent moiety that does not contain a region to which a target binding moiety binds. In some embodiments, a moiety of interest is an antibody agent that contains no Fc region. In some embodiments, a moiety of interest is or includes a scFv. In some embodiments, a scFv is for a different antigen than an antibody target agent.

In some embodiments, moieties of interest are or include reactive moieties, particularly those reaction partners for bio-orthogonal reactions. Suitable reactive moieties, including those for bio-orthogonal reactions, are widely known in the art and can be utilized herein. In some embodiments, a bio-orthogonal reaction is a cycloaddition reaction, e.g., click chemistry. In some embodiments, a moiety of interest is or includes —N3. In some embodiments, a moiety of interest is or includes an alkyne.

In some embodiments, a moiety of interest may be a moiety that binds to a SARS-COV-2 virus that is implicated in the COVID-19 disease. For example, the moiety that binds to a SARS-COV-2 virus may be a polypeptide disclosed in L. Cao et al., “De novo design of picomolar SARS-COV-2 mini-protein inhibitors” Science 370, 426-431 (2020), which is incorporated herein in its entirety by reference. Such a polypeptide moiety may result in binding to SARS-COV-2 spike proteins, inhibition, reduction and prevention of binding and/or infection of cells, inhibition, killing, and removal of SARS-CoV-2 viruses and/or cells infected thereby, etc. Various moieties of interest that interact with the SARS-COV-2 virus are described in International Patent Application No. PCT/US21/24186 filed Mar. 25, 2021, U.S. Provisional Patent Application No. 63/146,584 filed Feb. 6, 2021, and U.S. Provisional Patent Application No. 63/182,098 filed Apr. 30, 2021, each of which applications is incorporated herein in its entirety by reference.

In some embodiments, a moiety of interest improves one or more properties and/or activities of a target agent. In some embodiments, a moiety of interest is or includes a stability enhancer. In some embodiments, a moiety of interest improves one or more pharmacodynamic and/or pharmacokinetic properties of a target agent.

In some embodiments, at least one of the following conditions is met:
(a) the moiety of interest is or includes a therapeutic agent;
(b) the moiety of interest is or includes a moiety that can bind to a protein, nucleic acid or a cell; and/or
(c) the moiety of interest is or includes a reactive moiety suitable for a bio-orthogonal reaction.

In some embodiments, MOI is or includes a therapeutic agent moiety; and/or MOI is or includes an antibody agent.

### Linking Groups

In some embodiments, moieties are optionally connected to each other through linker moieties. For example, in some embodiments, a reactive group, e.g., RG, is connected to a moiety of interest, e.g., MOI, through a linker, e.g., L^{RM}. In some embodiments, a moiety, e.g., LG, may also include one or more linkers, e.g., L^{LG1}, L^{LG2}, L^{LG3}, L^{LG4}, etc., to link various portions. In some embodiments, L^{LG} is a linker moiety described herein. In some embodiments, L^{LG1} is a linker moiety described herein. In some embodiments, L^{LG2} is a linker moiety described herein. In some embodiments, L^{LG3} is a linker moiety described herein. In some embodiments, L^{LG4} is a linker moiety described herein. In some embodiments, L^{RM} is a linker moiety described herein. In some embodiments, L^{PM} is L as described herein. In some embodiments, L^{PM} is a linker moiety described herein. In some embodiments, L^{PM} is L as described herein.

Linker moieties of various types and/or for various purposes, e.g., those utilized in antibody-drug conjugates, etc., may be utilized in accordance with the present disclosure.

Linker moieties can be either bivalent or polyvalent depending on how they are used. In some embodiments, a linker moiety is bivalent. In some embodiments, a linker is polyvalent and connecting more than two moieties.

In some embodiments, L^{LM} includes one or more —[(CH₂)ₙ—O]ₘ—, wherein each n is independently 1-20, and m is 1-100.

In some embodiments, L^{RM} the linker includes one or more —[(CH₂)ₙ—O]ₘ—, wherein each n is independently 1-20, and m is 1-100.

### Reactive Groups

In some embodiments, provided compounds, e.g., those useful as reaction partners, include reactive groups (e.g., RG). As exemplified herein, in many embodiments, in provided compounds reactive groups (e.g., RG) are located between first groups (e.g., LG) and moieties of interest (e.g., MOI), and are optionally and independently linked to first groups and moieties of interest via linkers. In some embodiments, RG is a reaction group as described herein.

In some embodiments, as demonstrated herein, reactive groups when utilized in compounds that include no target binding moieties react slowly and provide low level of, in some embodiments, substantially no conjugation of moieties of interest with target agents. As demonstrated herein, combination of reactive groups with target binding moieties in the same compounds, e.g., as in compounds of formula R-I or salts thereof, can, among other things, promote reactions between reactive groups and target agents, enhance reaction efficiency, reduce side reactions, and/or improve reaction selectivity (e.g., in terms of target sites wherein conjugation of moieties of interest with target agents occurs).

Reactive groups in provided compounds can react with various types of groups in target agents. In some embodiments, reactive groups in provided compounds selectively react with amino groups of target agents, e.g., —NH₂ groups on side chains of lysine residues of proteins. In some embodiments, reactive groups when utilized in provided compounds, e.g., those of formula R-I or salts thereof, selectively react with particular sites of target agents, e.g., as shown in examples herein, one or more of K246, K248, K288, K290, K317, etc. of IgG1, K251, K253, etc. for IgG2, K239, K241 for IgG4, etc. In some embodiments, a site is K246 or K248 of an antibody heavy chain. In some embodiments, sites are K246 and/or K248 of an antibody heavy chain. In some embodiments, a site is K246 of an antibody heavy chain. In some embodiments, a site is K248 of an antibody heavy chain. In some embodiments, a site is K288 or K290 of an antibody heavy chain. In some embodiments, a site is K288 of an antibody heavy chain. In some embodiments, a site is K290 of an antibody heavy chain. In some embodiments, a site is K317. In some embodiments, a site is K414 of an antibody heavy chain. In some embodiments, a site is K185 of an antibody light chain. In some embodiments, a site is K187 of an antibody light chain. In some embodiments, sites are K251 and/or K253 of an IgG2 heavy chain. In some embodiments, a site is K251 of an IgG2 heavy chain. In some embodiments, a site is K253 of an IgG2 heavy chain. In some embodiments, sites are K239 and/or K241 of an IgG4 heavy chain. In some embodiments, a site is K239 of an IgG4 heavy chain. In some embodiments, a site is K241 of an IgG4 heavy chain. In some embodiments, conjugation selectively occurs at one or more heavy chain sites over light chain sites. In some embodiments, for technologies without target binding moieties, conjugation occurs at light chain sites more than heavy chain sites (e.g., see FIG. 15).

In some embodiments, a reactive group, e.g., RG, is or includes an ester group. In some embodiments, a reactive group, e.g., RG, is or includes an electrophilic group, e.g., a Michael acceptor.

In some embodiments, RG is a group of the formula -L^{LG2}, -L^{LG2}-L^{LG3}-L^{LG4}-L^{RG1}- or -L^{RG1}-L^{RG2}-, wherein:
L^{LG2} is —NH—, —NHC(O)—,—(CH₂)ₙ—NHC(O)—, —(CH₂)ₙ—OC(O)—, —(CH₂)ₙ—OC(O)NH—, —C(O)—NHCH₂—, —C(O)—NHCH₂CH₂—, —C(O)O—CH₂—, or NH—C(O)O—CH₂—;
L^{LG3} is an optionally substituted aryl ring;
L^{LG4-} is a bond, —NH—or —O—;
L^{RG1} is —O—C(O)—, —C(O)—, —S(O)—, —OS(O)₂—, or —OP(O(OR)—;
L^{RG2} is —CH₂—C(O)—, —C(O)—, or —CH₂—;
L^{LG} is —(O)C—[(CH₂)ₙO]ₘ(CH₂)ₙNH—, —(O)C—[(CH₂)ₙO]ₘ(CH₂)ₙNH—, —[(CH₂)ₙO]ₘNHC(O)[(CH₂)ₙO]ₘNH—, —[(CH₂)ₙO]ₘ{NHC(O)[(CH₂)ₙO]ₘ}ₚNH—, —[(CH₂)ₙO]ₘCy[(CH₂)ₙO]ₘNH—, —[(CH₂)ₙO]ₘCy[(CH₂)ₙO]ₘNHC(O)[(CH₂)ₙO]ₘNH—, or —[CH₂)ₙO]ₘCy[(CH₂)ₙO]ₘ{NHC(O)[(CH₂)ₙO]ₘ}ₚNH—,
wherein n, m, and p are integers independently chosen at each occurrence from 1-12, and Cy is an optionally substituted cyclic group.

In some embodiments, RG is a group of the formula -L^{LG2}-L^{LG3}-L^{LG4}-L^{RG1}- and is selected from:

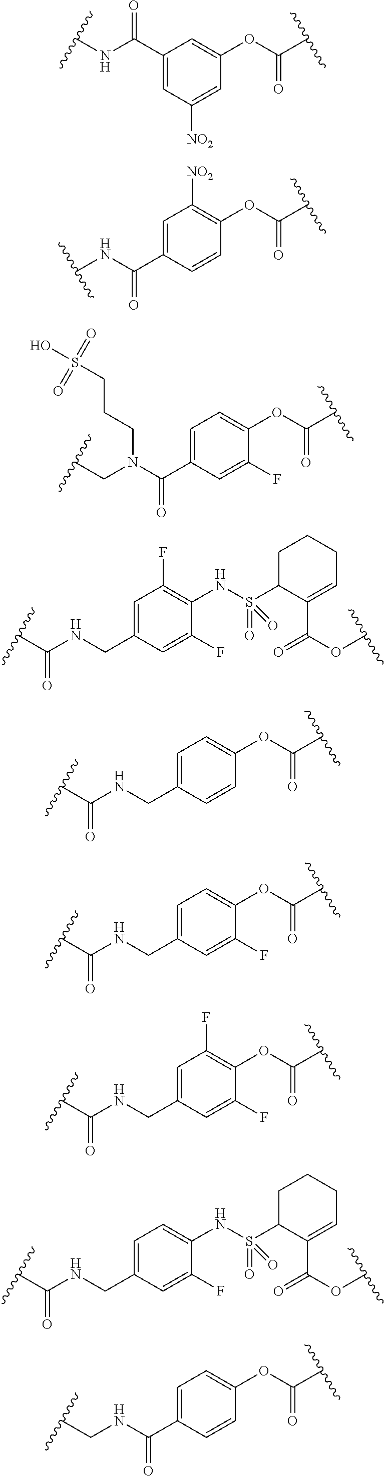

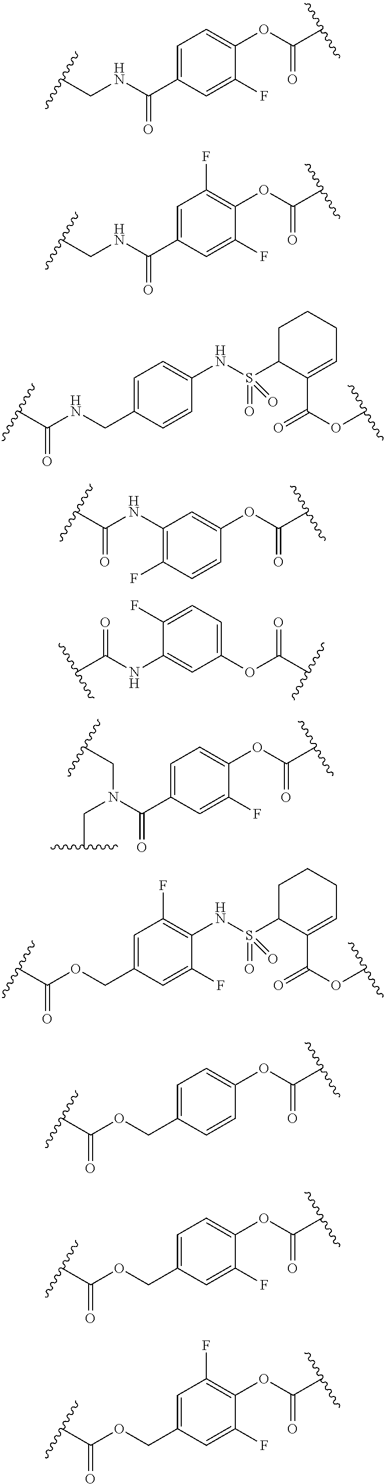

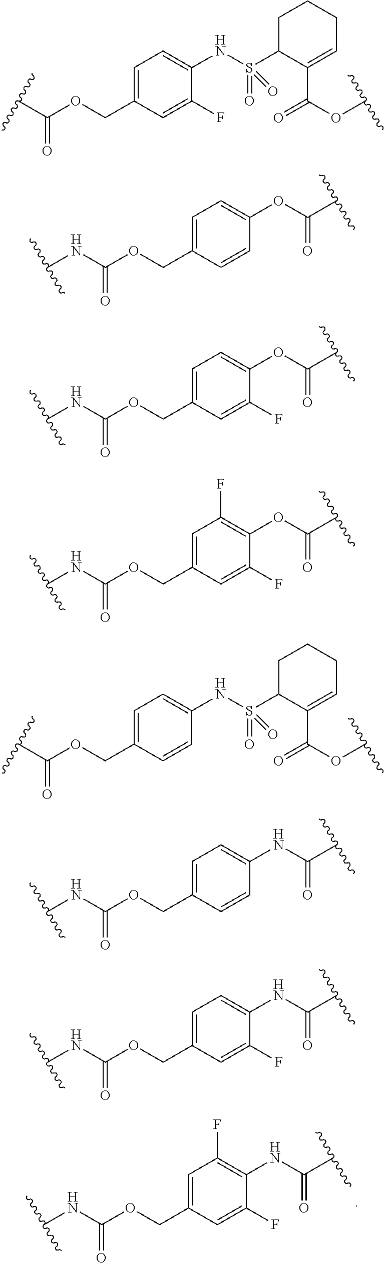

In some embodiments, n is 2 at each occurrence.

In some embodiments, the reactive group is or includes —C(O)—O— or —O—C(O)—.

In some embodiments, the reactive group includes an aryl group, optionally bonded to —C(O)—O— or —O—C(O)— and substituted with one or more electron-withdrawing groups.

In some embodiments, the aryl group has the structure of

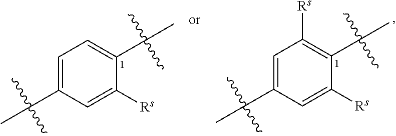

wherein R⁵ is independently chosen at each occurrence from halogen, —NO₂, —F, —L—R′, —C(O)—L—R′, —S(O)—L—R′, —S(O)₂—L—R′, and —P(O)(—L—R′)₂, and R′ is H or C₁-C₆ alkyl.

### Compositions

In some embodiment, the compositions may include the first and second compounds in equimolar amount. In some embodiments, the amount of the second compound may be 50 mole percent (mole %) or less based on the total number of moles of the first and second compounds in the composition. In some embodiments, the amount of the second compound may be 50 mole % or less, 45 mole % or less, 40 mole % or less, 35 mole % or less, 30 mole % or less, 25 mole % or less, 20 mole % or less, 15 mole % or less, 10 mole % or less, or 5 mole % or less based on the total number of moles of the first and second compounds in the composition. In some embodiments, the amount of the second compound may be 5% or less, 4% or less, 3% or less, 2% or less, or 1% or less based on the total number of moles of the first and second compounds in the composition. In some embodiments, the amount of the second compound may be 1.0% or less, 0.9% or less, 0.8% or less, 0.7% or less, 0.6% or less, 0.5% or less, 0.4% or less, 0.3% or less, 0.2% or less, 0.1% or less based on the total number of moles of the first and second compounds in the composition. In some embodiments, the amount of the second compound may be 0.10% or less, 0.09% or less, 0.08% or less, 0.07% or less, 0.06% or less, 0.05% or less, 0.04% or less, 0.03% or less, 0.02% or less, 0.01% or less based on the total number of moles of the first and second compounds in the composition. In some embodiments, the amount of the second compound may be 0.010% or less, 0.009% or less, 0.008% or less, 0.007% or less, 0.006% or less, 0.005% or less, 0.004% or less, 0.003% or less, 0.002% or less, 0.001% or less based on the total number of moles of the first and second compounds in the composition. In some embodiments, the amount of the second compound may be 0.0010% or less, 0.0009% or less, 0.0008% or less, 0.0007% or less, 0.0006% or less, 0.0005% or less, 0.0004% or less, 0.0003% or less, 0.0002% or less, 0.0001% or less based on the total number of moles of the first and second compounds in the composition. In some embodiments, the amount of the second compound may be 0.00010% or less, 0.00009% or less, 0.00008% or less, 0.00007% or less, 0.00006% or less, 0.00005% or less, 0.00004% or less, 0.00003% or less, 0.00002% or less, 0.00001% or less based on the total number of moles of the first and second compounds in the composition. In some embodiments, the amount of the second compound may be 0.000010% or less, 0.000009% or less, 0.000008% or less, 0.000007% or less, 0.000006% or less, 0.000005% or less, 0.000004% or less, 0.000003% or less, 0.000002% or less, 0.000001% or less based on the total number of moles of the first and second compounds in the composition.

In some embodiment, the compositions may further include a third compound, a fourth compound, or a combination thereof. In some embodiments, the amount of the third compound, the fourth compound, or the combination thereof may be 5% or less, 4% or less, 3% or less, 2% or less, or 1% or less based on the number of moles of the first compound in the composition. In some embodiments, the amount of the third compound, the fourth compound, or the combination thereof may be 1.0% or less, 0.9% or less, 0.8% or less, 0.7% or less, 0.6% or less, 0.5% or less, 0.4% or less, 0.3% or less, 0.2% or less, 0.1% or less based on the number of moles of the first compound in the composition. In some embodiments, the amount of the third compound, the fourth compound, or the combination thereof may be 0.10% or less, 0.09% or less, 0.08% or less, 0.07% or less, 0.06% or less, 0.05% or less, 0.04% or less, 0.03% or less, 0.02% or less, 0.01% or less based on the number of moles of the first compound in the composition. In some embodiments, the amount of the third compound, the fourth compound, or the combination thereof may be 0.010% or less, 0.009% or less, 0.008% or less, 0.007% or less, 0.006% or less, 0.005% or less, 0.004% or less, 0.003% or less, 0.002% or less, 0.001% or less based on the number of moles of the first compound in the composition. In some embodiments, the amount of the third compound, the fourth compound, or the combination thereof may be 0.0010% or less, 0.0009% or less, 0.0008% or less, 0.0007% or less, 0.0006% or less, 0.0005% or less, 0.0004% or less, 0.0003% or less, 0.0002% or less, 0.0001% or less based on the number of moles of the first compound in the composition. In some embodiments, the amount of the third compound, the fourth compound, or the combination thereof may be 0.00010% or less, 0.00009% or less, 0.00008% or less, 0.00007% or less, 0.00006% or less, 0.00005% or less, 0.00004% or less, 0.00003% or less, 0.00002% or less, 0.00001% or less based on the number of moles of the first compound in the composition. In some embodiments, the amount of the third compound, the fourth compound, or the combination thereof may be 0.000010% or less, 0.000009% or less, 0.000008% or less, 0.000007% or less, 0.000006% or less, 0.000005% or less, 0.000004% or less, 0.000003% or less, 0.000002% or less, 0.000001% or less based on the number of moles of the first compound in the composition.

In another embodiment, provided is a composition including:

a first compound having the structure of formula (P-II):

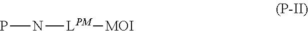

wherein:
P-N is a protein agent moiety including a lysine residue;
L^{PM} is a linker; and
MOI is a moiety of interest; and at least one of
a second compound having the structure:

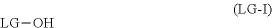

wherein LG is a group including a target binding moiety that binds to a target agent; and a third compound having the formula (R-I):

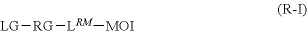

LG is a group including a target binding moiety that binds to a target agent, which is identical to LG in formula (LG-I);
RG is a reactive group;
L^{RM} is a linker, which is identical to in formula (P-II); and
MOI is a moiety of interest.

In another embodiment, the composition may further include:
a fourth compound having the formula (R-III):

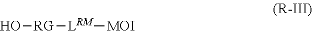

or a combination thereof.

The invention is further illustrated by the following non-limiting examples.

### EXAMPLES

### Example 1. Certain Technologies for Preparing Agents: I-29, I-30, I-31, I-32, I-33, I-34, I-35, I-36.

In some embodiments, the present disclosure provides technologies for preparing MATE agents and compositions thereof. In some embodiments, provided technologies include reacting a composition including a plurality of antibody agents (e.g., IVIG compositions such as Gamunex-C) with a composition including a plurality of agents each including a target binding moiety, an antibody binding moiety, and a reactive group in between (and optional linker moieties linking such moieties) (e.g., I-3, I-7, I-8, I-15, I-19, I-20, I-21, I-22, I-23, etc.). Described below as examples are preparations of certain MATE agents and compositions thereof.

### 1. A Reaction/Conjugation Protocol

### 1.1 IVIG Buffer Exchange

Gamunex-C (1 mL, ˜100 mg/mL) was buffer exchanged into 50 mM borate pH 8.2 using Amicon-15 mL unit with MWCO 30 kDa. The protein concentration of the buffer exchanged IVIG was determined by UV-Vis using extinction coefficient of 1.41 mL·mg−1cm−1 at 280 nm. The buffer exchanged IVIG was adjusted to 20 mg/ml using 50 mM borate buffer pH 8.2.

### 1.2 Reagent Stock Solution Preparation

Reagents were weighed individually and the DMSO volume used to prepare the stock solution is calculated as in the following:

DMSO volume (mL)=(Solid weight (mg)/Molecular weight)×purity (%)/5 mM×10⁶.

Data for certain preparations of reagents are presented below:

| | Solid | | | DMSO | |
|---|---|---|---|---|---|
| | amount | Purity | Molecular | volume | Final reagent |
| Reagent | (mg) | (%) | weight | (μL) | concentration |
| I-8 | 4.05 | 77.1 | 9159.31 | 68.2 | 5 mM |
| I-7 | 2.17 | 87.1 | 9511.73 | 39.7 | |
| I-15 | 1.85 | 86.0 | 9510.75 | 33.4 | |
| I-19 | 1.44 | 86.4 | 10084.38 | 24.7 | |
| I-21 | 0.65 | 60.3 | 9049.24 | 8.7 | |
| I-22 | 2.42 | 91.8 | 9472.74 | 46.9 | |
| I-23 | 1.50 | 91.5 | 9091.28 | 30.2 | |
| I-20 | 2.39 | 97.9 | 9514.78 | 49.2 | |

The structures of the Reagents I-7, I-8, I-15, I-19, I-20, I-21, I-22, and I-23 are shown below:

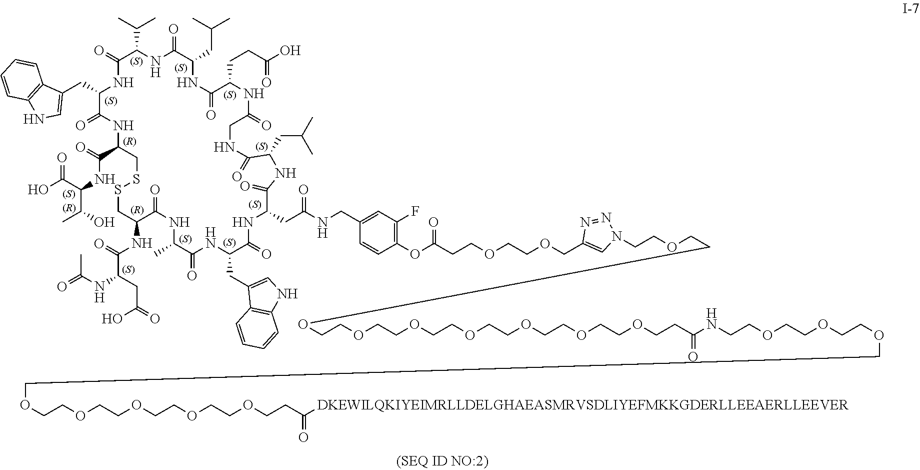

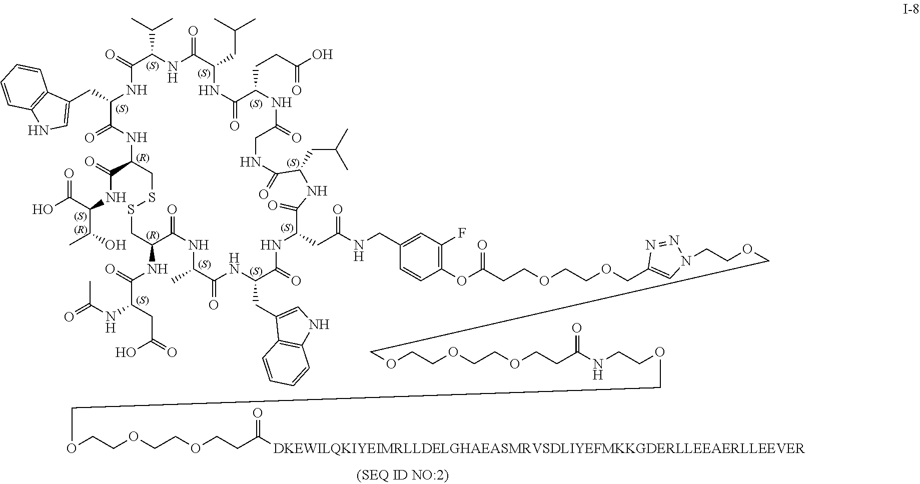

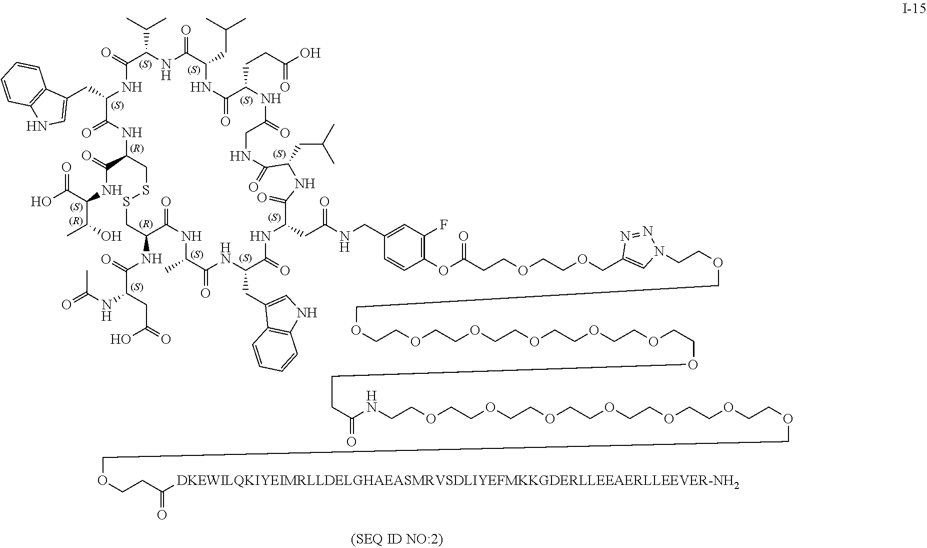

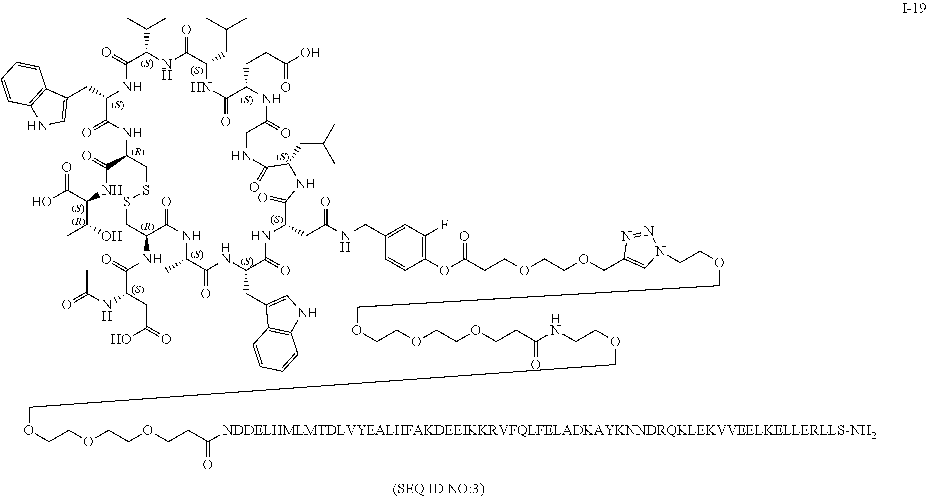

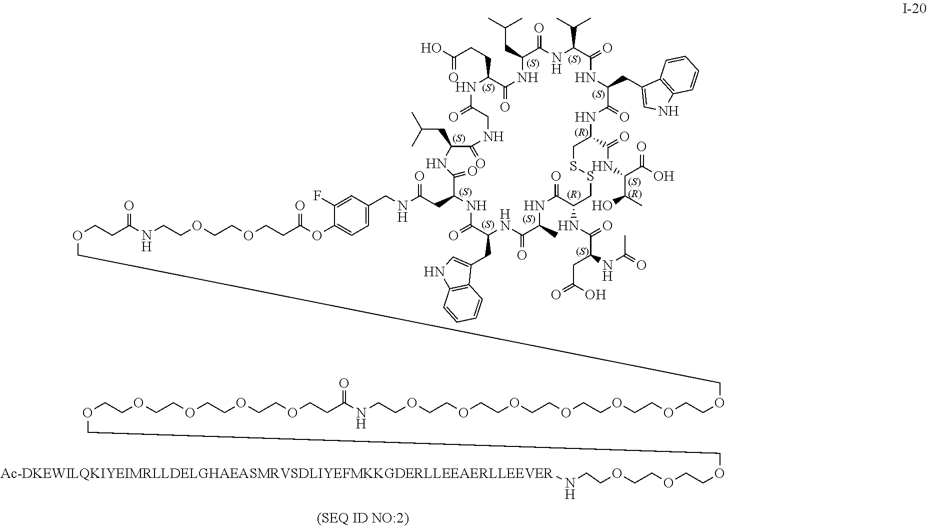

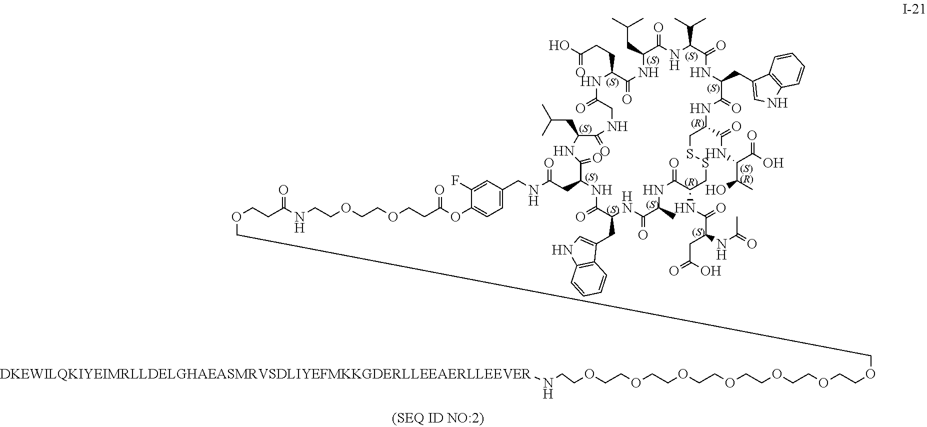

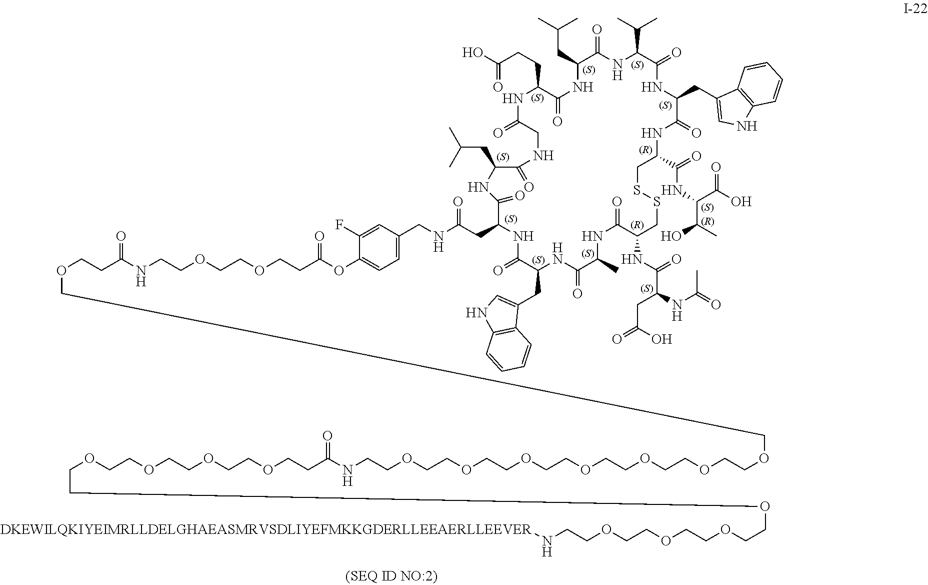

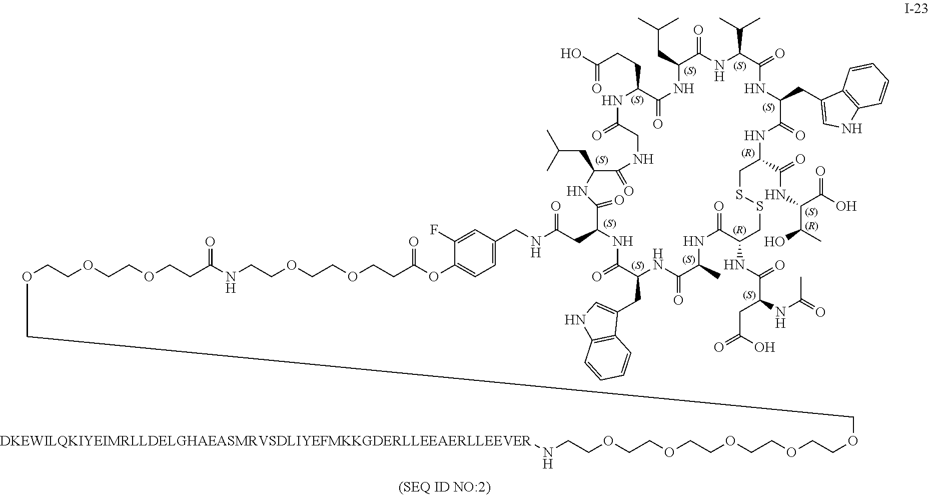

### 1.3 Conjugation Reaction Setup

Procedure for I-29, I-30, I-31, I-32, I-34, I-35, I-36:

To IVIG in 50 mM borate pH 8.2 (4 mg, 20 mg/mL, 200 μL) was added reagent (5 mM in DMSO, 13.3 μL, 2.5 eq.). The reaction was mixed thoroughly and rotated at room temperature overnight.

Procedure for I-33:

To IVIG in 50 mM borate pH 8.2 (2.62 mg, 20 mg/mL, 130.8 μL) was added reagent (5 mM in DMSO, 8.7 μL, 2.5 eq.). The reaction was mixed thoroughly and rotated at room temperature overnight. The following table shows the MATE reagents used to produce each of the MATE reagents discussed in further detail in the following section.

| MATE Reagent | MATE Product |
|---|---|
| I-8 | I-29 |
| I-7 | I-30 |
| I-15 | I-31 |
| I-19 | I-32 |
| I-22 | I-34 |
| I-23 | I-35 |
| I-20 | I-36 |

In each of the above procedures, in addition to the agent I-29, I-30, I-31, I-32, I-33, I-34, I-35, I-36, the reaction mixtures included the following product, which was formed in equimolar amount to the agent I-29, I-30, I-31, I-32, I-33, I-34, I-35, and I-36:

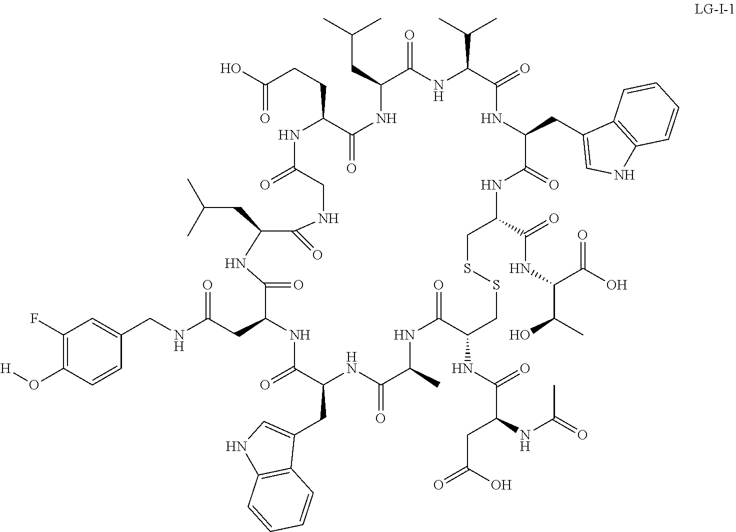 [SMILES: CC(=O)N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](CC(=O)NCc2ccc(O)c(F)c2)NC(=O)[C@H](Cc2c[nH]c3ccccc23)NC(=O)[C@H](C)NC1=O]

### 2. Purification

A crude mixture was buffer exchanged with 50 mM glycine pH 2.2 in Amicon-4 mL MWCO 30 kDa for 2 cycles. Then it was buffer exchanged into PBS in Amicon-4 mL MWCO 30 kDa for 2 cycles. Insoluble white precipitates were observed at the bottom of the filter. In some embodiments, released antibody binding moieties were removed under acidic conditions.

Solution and precipitates from the eight samples were collected in 1.5 ml tube, respectively, and centrifuged at 16000 g for 5 min. The clear supernatant was carefully collected. The concentration of protein conjugates in supernatant was determined by UV-Vis Nanodrop instrument using extinction coefficient 1.41 mL·mg−1cm−1. Concentration, volume, and yield of protein conjugates from certain preparations are shown in the table below.

| | Concentration | Volume | Product mass | Yield |
|---|---|---|---|---|
| Agent | (mg/mL) | (mL) | (mg) | (%) |
| I-29 | 6.29 | 0.501 | 3.15 | 79 |
| I-30 | 6.86 | 0.45 | 3.09 | 77 |
| I-31 | 2.25 | 0.667 | 1.5 | 38 |
| I-32 | 4.83 | 0.695 | 3.36 | 84 |
| I-33 | 5.55 | 0.548 | 3.04 | 116 |
| I-34 | 7.35 | 0.365 | 2.68 | 67 |
| I-35 | 10.7 | 0.326 | 3.49 | 87 |
| I-36 | 3.03 | 0.707 | 2.14 | 53 |

To adjust the final MATE agent concentration to 1.5-3.0 mg/ml range, all the conjugates were diluted with PBS to target for 3.0 mg/mL. In some embodiments, it was observed that, upon adding PBS, white precipitation quickly formed in some or all samples. To recover the conjugates, those samples were centrifuged at 16000 g for 3 minutes. The clear supernatants were carefully collected. The concentration of protein conjugates in supernatant was determined by UV-Vis Nanodrop instrument using extinction coefficient 1.41 mL·mg−1cm−1. The concentration, volume, and yield of the final conjugates from certain preparations are summarized in the table below.

| | Concentration | Volume | Product mass | Yield |
|---|---|---|---|---|
| Agent | (mg/mL) | (mL) | (mg) | (%) |
| I-29 | 1.58 | 0.66 | 1.04 | 26 |
| I-30 | 1.52 | 0.92 | 1.39 | 35 |
| I-31 | 2.25 | 0.667 | 1.5 | 38 |
| I-32 | 2.15 | 1.00 | 2.15 | 54 |
| I-33 | 2.15 | 0.69 | 1.16 | 44 |
| I-34 | 1.62 | 0.79 | 1.28 | 27 |
| I-35 | 2.48 | 0.64 | 1.58 | 40 |
| I-36 | 3.03 | 0.707 | 2.14 | 53 |

### 3. Analytical Method for DAR (Ratio of Target Binding Moiety/Antibody Moiety) Determination

MATE conjugate (50 μg) was incubated with 10× glycol-buffer 2 (0.1×total volume), 1 μL IdeZ and 1 μL PNGase F at 37° C. for 1 hour. Then 8 μg of the sample was taken out and diluted 50× with water. The injection volume was 2 μL or 3 μL, depending on the desired signal.
Instrument:
LC: Waters Acquity UPLC protein BEH C4 column (300 Å, 17 μm×2.1 mm×50 mm)
MS: Waters Xevo G2-QTOF
Processing Software: MassLynx
Mobile A: water+0.1% formic acid
Mobile B: acetonitrile+0.1% formic acid

Data analysis: A total ion chromatogram (TIC) is obtained. A TIC region is selected for spectral analysis. The region is selected to encompass both conjugated and unconjugated Fc with no bias. A MS profile is obtained from the TIC. A charge state envelope is selected for deconvolution. The raw spectrum is deconvoluted using MaxEnt1 deconvolution tool to obtain the zero charge spectrum. BAR (Binder distribution ratio in Fc×Fc per antibody) is calculated using the following formula:

$$BAR = [ 1 ( {Fc}_{conj )} ) / [ 1 ( {Fc}_{conj} ) + 1 ( {Fc}_{unconjug} ) ] \times 2$$

Deconvolute m/z 8000-30000, resolution 1.50. The results of DAR by LC-MS analysis are shown below.

| | DAR of crude | DAR of purified |
|---|---|---|
| Agent | conjugates | conjugates |
| I-29 | 1.08 | 1.47 |
| I-30 | 1.64 | 1.78 |
| I-31 | 1.75 | 1.72 |
| I-32 | 0.90 | 1.21 |
| I-33 | 1.81 | 1.73 |
| I-34 | 1.58 | 1.77 |
| I-35 | 1.76 | 1.76 |
| I-36 | 1.49 | 1.79 |

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art.

Those skilled in the art will recognize, or be able to ascertain using no more than routine experimentation, numerous equivalents to the specific procedures described herein. Such equivalents are considered to be within the scope of this disclosure and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

### Example 2. Analytical Methods for Determining Drug Antibody Ratio (DAR), DAR Distribution, and MATE-Reagent Related Impurities

### a. Quantification of DAR Species and Identification of Impurities

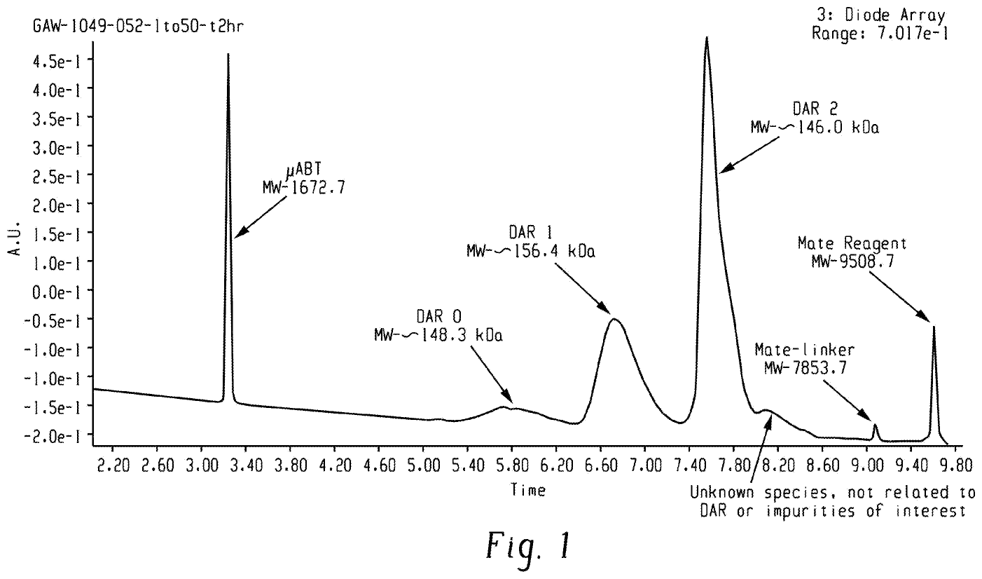

The following UPLC analytical method allows simultaneous determination of species with different DARs and MATE-reagent related impurities. Drug substance/ Drug Product (DR/DP) and DAR/DAR distribution is calculated based on individual DAR component HPLC/UV280 nm signal area. MATE-reagent related DS/DP impurity content (MATE-reagent, MATE-linker and universal antibody binding terminal (uABT)) is determined as a ratio of sample impurity concentration to DS/DP protein concentration (reported as % wt. impurity/wt. protein). The identity of UPLC signals was confirmed by protein and individual impurity RS and LCMS analysis. A representative UPLC/UV280 Trace is shown for compound I-36 in FIG. 1. DS is the desired product (MATE), and the impurity is expressed as a weight % of that product (DS/DP).

The UPLC method is performed with a WATERS ACQUITY UPLC system, UV 280 nm detection. The MS detector is a WATERS XEVO G2-XS QTof detector. This detector is used for species molecular weight determination. The autosampler functions at ambient conditions, 22-23° C. A HALO 1000 Å Diphenyl column, 2.7 μm, 2.1×50 mm with a column temperature of 80° C. is used. The flow rate is 0.5 mL/min. Mobile phase A is 0.5% trifluoroacetic acid; mobile phase B is acetonitrile with 0.05% trifluoroacetic acid.

TABLE 1
UPLC Gradient

| Time | Flow (mL/min.) | % B |
|---|---|---|
| 0 | 0.5 | 10 |
| 1 | 0.5 | 20 |
| 10 | 0.5 | 50 |
| 10.1 | 1.0 | 100 |
| 11 | 1.0 | 100 |
| 11.1 | 1.0 | 10 |
| 12 | 0.5 | 10 |

The MS detector settings for the Waters Xevo G2-XS QTof detector for the Impurity Analysis and DAR Analysis methods are shown in Table 2.

TABLE 2
MS Detector Settings

| | MS Method Parameter Value | | | |
|---|---|---|---|---|
| Parameter | Impurity Analysis | | DAR Analysis | |
| Ionization Mode | Electrospray Ionization (ESI), positive mode | | | |
| Detection | Time of flight | | | |
| Scan range | 500-3000 | | | |
| Capillary Voltage | +2 | kV | +1.5 | kV |
| Sampling Cone | 100 | | 195 | |
| Source Offset | 80 | | 150 | |
| Source Temperature | 150° | C. | 150° | C. |
| Desolvation Temperature | 500° | C. | 650° | C. |
| Cone Gas | 50 | L/h | 100 | L/h |
| Desolvation Gas | 800 | L/h | 1000 | L/h |

The DS/DP sample is diluted to a protein concentration of approximately 1.5 mg/ml, corresponding to a 50-fold DS/DP dilution in a DMSO: water dilution vehicle, 6:94 v/v. Samples, such as I-36, stored −20° C. Thaw and equilibrate sample to ambient temperature for approximately 2 hours. Vortex sample for 1-2 minutes. Dilute to target concentration (protein 1.5 mg/mL) in dilution vehicle (DMSO:Water=6:94). Diluted sample remain at RT until analysis, which should be completed within 4 hours of sample dilution. Alternatively, diluted sample can be stored before analysis at 2-8° C. for 24 hours. If diluted sample is stored at 2-8° C. the sample should be allowed to equilibrate to RT for half-hour, and then vortexed for 1-2 minutes prior to use.

### b. I-36 Drug Antibody Ratio (DAR) and DAR Distribution Analysis

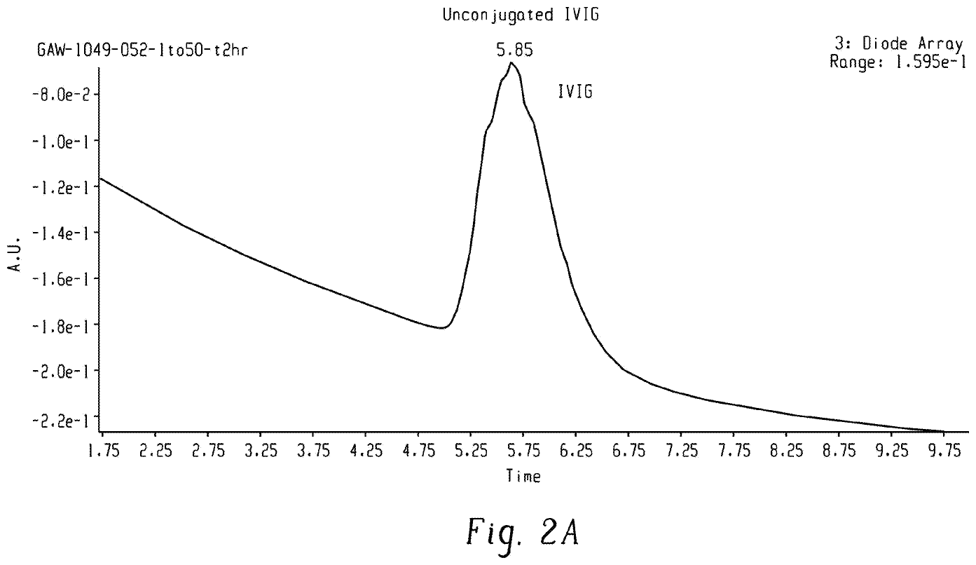

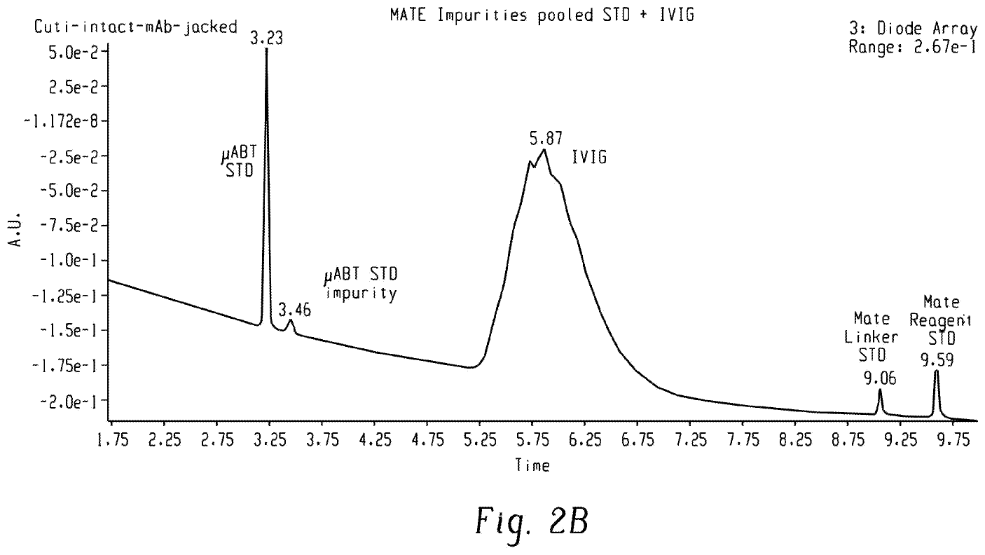

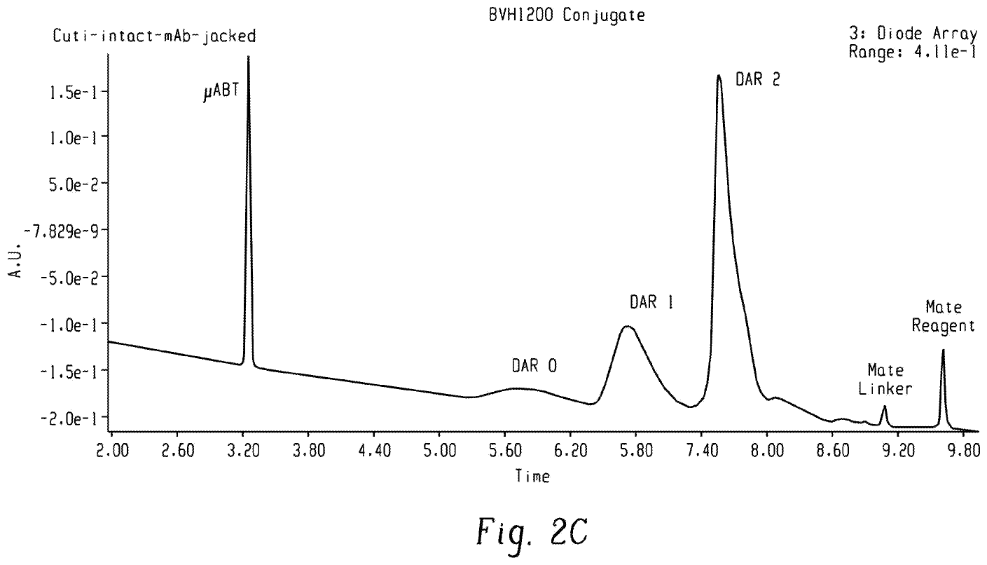

Using the UPLC method, detector parameters, and sample preparation methods given in the preceding section the DAR and DAR distribution are determined for I-36 preparations. Prior to quantitating DAR species UPLC spectra of (i) unconjugated IVIG, (ii) a pooled standard of MATE impurities with unconjugated IVIG are obtained. These spectra are shown in FIGS. 2A-C. Data is shown in Tables 3 and 4.

TABLE 3

| | | Analyte | | | Analyte | |
|---|---|---|---|---|---|---|
| | | conc. | | | conc. | |
| | | (% total | Ave. | | (% total | Ave. |
| # | Analyte | protein) | DAR | Analyte | protein) | DAR |
| 1 | Total IVIG | 100% | 1.55 | Total IVIG | 100% | 1.58 |
| | Protein | | | Protein | | |
| 2 | DAR-0 species | 6.2% | | DAR-0 species | 6.0% | |
| 3 | DAR-1 species | 32.8% | | DAR-1 species | 29.7% | |
| 4 | DAR-2 species | 61.1% | | DAR-2 species | 64.3% | |

TABLE 4
Deconvoluted MS Spectra for Individual
DAR Variants and Unconjugated IVIG

| | Retention Time | | Peak |
|---|---|---|---|
| Species | (min.) | MW | Intensity |
| Unconjugated IVIG | 5.70 | ~148.9 kDa | 148917.9 |
| I-36 DAR 0 | 5.70 | ~148.3 kDa | 148289.0 |
| I-36 DAR 1 | 6.64 | ~156.4 kDa | 156418.5 |
| | | (MW increase to | |
| | | IVIG ~7.5 kDa) | |
| I-36 DAR 2 | 7.43 | ~164.0 kDa | 156418.5 |
| | | (MW increase to | |
| | | IVIG ~15.1 kDa) | |

### b. UPLC Method for MATE Impurity Analysis(1) Calibration Curve Preparation

A pooled standard stock solution (PL STOCK) is prepared as follows. Individual uABT, MATE-Reagent (MR) and MATE-Linker standard (STD) stock solutions are prepared in DMSO at STD stock solution concentrations of 2.00 mg/ml. Equal volumes of individual 1 mg/mL DMSO stock solutions are combined. Resulting individual pooled stock solution concentration is 0.667 mg/mL (PL Stock). Store DMSO standard stock solutions at −70° C. for up to one month.

A calibration curve having an individual impurity concentration range 0.004 mg/ml to 0.04 mg/ml (or 0.02 ug/injection to 0.2 ug/injection for 5-ul injection volume) is needed to quantitate most impurities formed during the MATE reaction. DMSO solutions are thawed at ambient temperature, and then vortex thoroughly. IVIG working stock (82.5 mg/ml) is prepared by 2-fold dilution of 165 mg/mL IVIG reference material with water. Calibration curve standards are according to Table 5. 6% DMSO concentration and constant IVIG: Pooled Impurity ratio (w/w) is maintained for all calibrators. Calibrator solutions are stored at ambient temperature. Calibration solutions have limited stability; therefore, analysis should be performed within 4 hours after calibrator preparation.

TABLE 5
Pooled Calibration Standards

| | | | | IVIG | | |
|---|---|---|---|---|---|---|
| | PL | | | Stock, | | |
| | Stock | Pooled | | 82.5 | | Total |
| PL STD | (0.667 | STD | DMSO | mg/mL | Water | Volume |
| (mg/mL) | mg/mL) | (μl) | (μl) | (μl) | (μl) | (μl) |
| 0.04 | PL Stock | 18.0 | 0 | 15.0 | 267.0 | 300 |
| 0.03 | PL Stock | 13.5 | 4.5 | 11.3 | 270.8 | 300 |
| 0.02 | PL Stock | 9.0 | 9.0 | 7.5 | 274.5 | 300 |
| 0.01 | PL Stock | 4.5 | 13.5 | 3.8 | 278.3 | 300 |
| 0.004 | 10x DMSO | 18 | 0 | 1.5 | 280.5 | 300 |
| | diluted PL | | | | | |
| | Stock | | | | | |

### (2) DS/DP Sample Handling

Diluted DS/DP samples are stable for at least 2 hours. A 50-fold DS/DP sample dilution was selected for impurity analysis following the study of diluted sample reproducibility shown in Table 6.

TABLE 6

| Protein | Sam- | uABT | | MATE-Linker | | MATE Reagent | |
|---|---|---|---|---|---|---|---|
| Conc. | ple | (% wt./wt. | | (% wt./wt. | | (% wt./wt. | |
| (mg/ | dilu- | protein) | | protein) | | protein) | |
| mL) | tion | T₀ | 2 hrs | T₀ | 2 hrs | T₀ | 2 hrs |
| 7.50 | 10 | 1.76% | 1.76% | 0.74% | 0.76% | 3.19% | 3.12% |
| 1.50 | 50 | 1.64% | 1.60% | 0.68% | 0.67% | 2.84% | 2.78% |
| 0.75 | 100 | 1.57% | 1.59% | 0.64% | 0.58% | 2.79% | 2.63% |
| 0.50 | 150 | 1.57% | 1.58% | 0.62% | 0.53% | 2.78% | 2.54% |
| 0.38 | 200 | 1.56% | 1.57% | 0.58% | 0.56% | 2.74% | 2.61% |
| 0.30 | 250 | 1.56% | 1.59% | 0.56% | 0.44% | 2.72% | 2.41% |
| 0.25 | 300 | 1.66% | 1.67% | 0.53% | 0.51% | 2.78% | 2.57% |
| 0.30 | 250 | 1.56% | 1.59% | 0.56% | 0.44% | 2.72% | 2.41% |
| 0.25 | 300 | 1.66% | 1.67% | 0.53% | 0.51% | 2.78% | 2.57% |

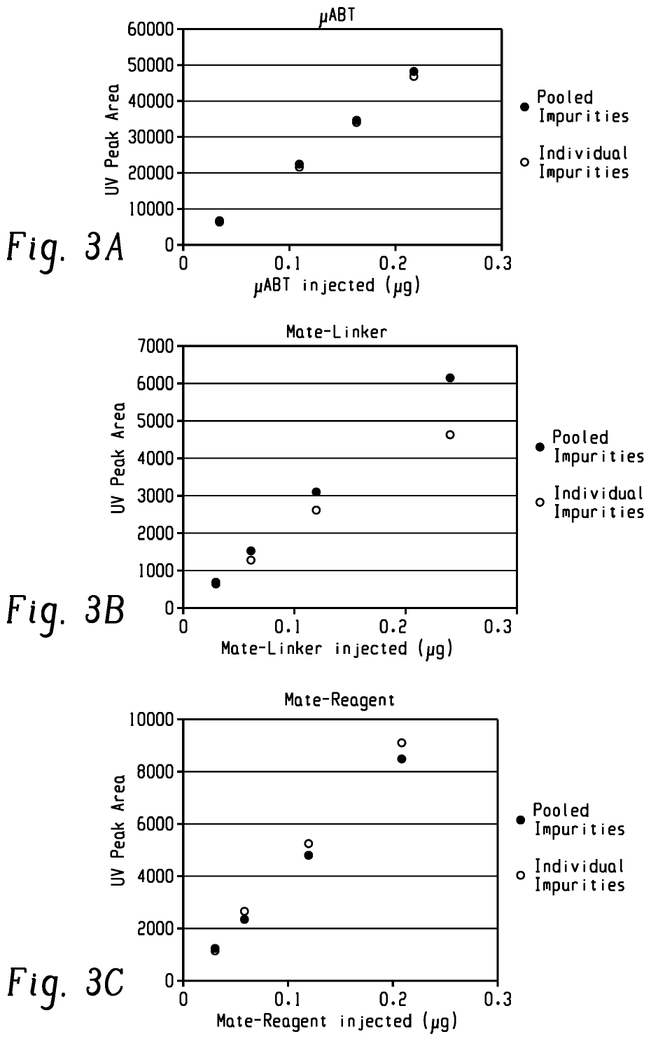

The calibration curve was found to be reproducible for individual and pooled impurities. Calibration curve data for uABT, MATE-Linker, and MATE-Reagent samples are shown in FIG. 3. The calibration curve calculators for uABT, MATE-Linker, and MATE-Reagent for I-36/I-20 were extracted from the calibration curves and are presented in Table 7.

TABLE 7

| | Individual Calibrators | | Pooled Calibrators | |
|---|---|---|---|---|
| | Linear | | Linear | |
| Impurity | Regression | R² | Regression | R² |
| uABT | y = 220251x − | 0.9997 | y = 223653x − | 0.9982 |
| | 2343.5 | | 1622 | |
| MATE-Linker | y = 22668x − | 1.0000 | y = 25971X − | 0.9998 |
| | 80.552 | | 55.739 | |
| MATE-Reagent | y = 40648x − | 0.9999 | y = 43591x − | 0.9999 |
| | 31.285 | | 8.3333 | |

Using the calibration curve calculators shown in Table 7, the impurities for two repetitions of the I-36 conjugation reaction are calculated. The results are in Table 8. The total protein concentration was established using the protein calibration curve.

TABLE 8

| | | Analyte Concentration | | |
|---|---|---|---|---|
| | Analyte | mg/mL | mg/mg protein | % wt./wt. protein |
| 1 | Total IVIG Protein | 57.79 | n/a | n/a |
| 2 | Free uABT | 0.93 | 0.0161 | 1.61% |
| 3 | Free MATE-reagent | 1.65 | 0.0286 | 2.86% |
| 4 | Free MATE-linker | 0.35 | 0.0061 | 0.61% |
| 1 | Total IVIG Protein | 64.52 | n/a | n/a |
| 2 | Free uABT | 1.13 | 0.0175 | 1.75% |
| 3 | Free MATE-reagent | 1.38 | 0.0214 | 2.14% |
| 4 | Free MATE-linker | 0.64 | 0.0099 | 0.99% |

1. A composition comprising:
a first compound having the structure of formula (P-II): 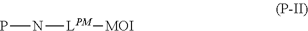
wherein:
P-N is a protein agent moiety comprising a lysine residue;
L^{PM} is a linker; and
MOI is a moiety of interest; and
a second compound having the structure: 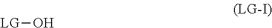
wherein LG is a group comprising a target binding moiety that binds to a target agent.

2. The composition of claim 1, further comprising:
a third compound having the formula (R-I): 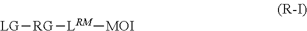
LG is a group comprising a target binding moiety that binds to a target agent, which is identical to LG in formula (LG-I);
RG is a reactive group;
L^{RM} is a linker, which is identical to in formula (P-II); and
MOI is a moiety of interest;
a fourth compound having the formula (R-III): 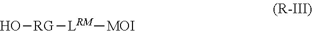
or a combination thereof.

3. The composition of claim 1 or 2, wherein
LG is R^{LG}-L^{LG};
R^{LG} is 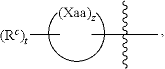
R^{c}-(Xaa)z-, a nucleic acid moiety, or a small molecule moiety;
each Xaa is independently a residue of an amino acid or an amino acid analog;
t is 0-50;
z is 1-50;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, -L^{LG1}L^{LG2}-, -L^{LG1}-L^{LG2}-L^{LG3}-, or -L^{LG1}-L^{LG2}-L^{LG3}-L^{LG4}-;
RG is -L^{RG1}-L^{RG2}-, -L^{LG4}-L^{RG1}-L^{RG2}-, -L^{LG3}-L^{LG4}-L^{RG1}-L^{RG2}-, or -L^{LG2}-L^{LG3}-L^{LG4}-L^{RG1}-L^{RG2}-;
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S——S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

4. The composition of claim 1 or 2, wherein LG is or comprises a target binding moiety that binds to a target agent, wherein the target agent is an antibody agent.

5. The composition of claim 3, wherein LG is or comprises a target binding moiety that binds to a Fc region, and/or R^{LG} is or comprises DCAWXLGELVWCT (SEQ ID NO:1), wherein the two cysteine residues optionally form a disulfide bond, and X is an amino acid residue.

6. The composition of claim 3, wherein at least one of the following conditions is met:
(a) the moiety of interest is or comprises a therapeutic agent;
(b) the moiety of interest is or comprises a moiety that can bind to a protein, nucleic acid or a cell; and/or
(c) the moiety of interest is or comprises a reactive moiety suitable for a bio-orthogonal reaction.

7. The composition of claim 1 or 2, wherein LG is or comprises a target binding moiety having the structure of formula A-1 to A-50 shown in the specification.

8. The composition of claim 1 or 2, wherein MOI is or comprises a therapeutic agent moiety and/or MOI is or comprises an antibody agent.

9. The composition of claim 1, wherein L^{LM} comprises one or more —[(CH₂)ₙ—O]ₘ—, wherein each n is independently 1-20, and m is 1-100.

10. The composition of claim 2, wherein L^{RM} the linker comprises one or more —[(CH₂)ₙ—O]ₘ—, wherein each n is independently 1-20, and m is 1-100.

11. The composition of claim 1 or 2, wherein RG is a group of the formula -L^{LG2}, -L^{LG2}-L^{LG3}-L^{LG4}-L^{RG1}- or --L^{RG1}-L^{RG2}-, wherein
L^{LG2} is —NH—, —NHC(O)—, —(CH₂)ₙ—NHC(O)—, —(CH₂)ₙ—OC(O)—, —(CH₂)ₙ—OC(O)NH—, —C(O)—NHCH₂—, —C(O)—NHCH₂CH₂—, —C(O)O—CH₂—, or —NH—C(O)O—CH₂—;
L^{LG3} is an optionally substituted aryl ring;
L^{LG4-} is a bond, —NH— or —O—;
L^{RG1} is —O—C(O)—, —C(O)—, —S(O)—, —OS(O)₂—, or —OP(O(OR)—;
L^{RG2} is —CH₂—C(O)—, —C(O)—, or —CH₂—;
LG is —(O)C—[(CH₂)ₙO]ₘ(CH₂)ₙNH—, —(O)C—[(CH₂)ₙO]ₘ(CH₂)ₙNH—, —[(CH₂)ₙO]ₘNHC(O)[CH₂)ₙO]ₘNH—, —[(CH₂)ₙO]ₘ{NHC(O)[(CH₂)ₙO]ₘ}ₚNH—, —[(CH₂)ₙO]ₘCy[(CH₂)ₙO]ₘNH—, —[(CH₂)ₙO]ₘCy[(CH₂)ₙO]ₘNHC(O)[(CH₂)ₙO]ₘNH—, or —[(CH₂)ₙO]ₘCy[(CH₂)ₙO]ₘ{NHC(O)[(CH₂)ₙO]ₘ}ₚNH—,
wherein n, m, and p are integers independently chosen at each occurrence from 1-12, and Cy is an optionally substituted cyclic group.

12. The composition of claim 11, wherein RG is a group of the formula -L^{LG2}-L^{LG3}-L^{LG4}-L^{RG1}- and is selected from:

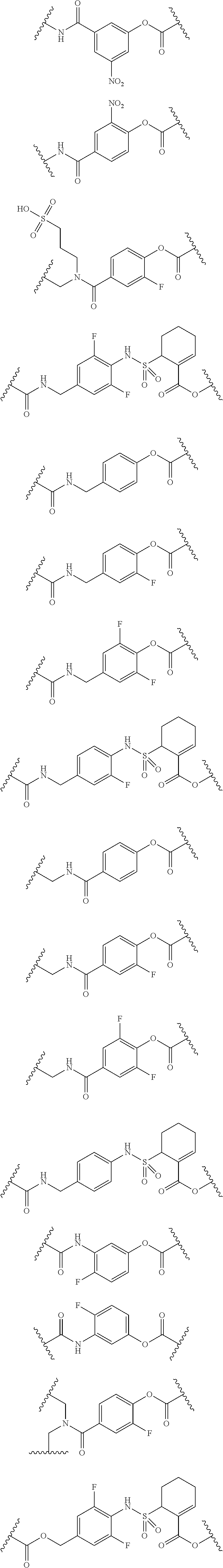

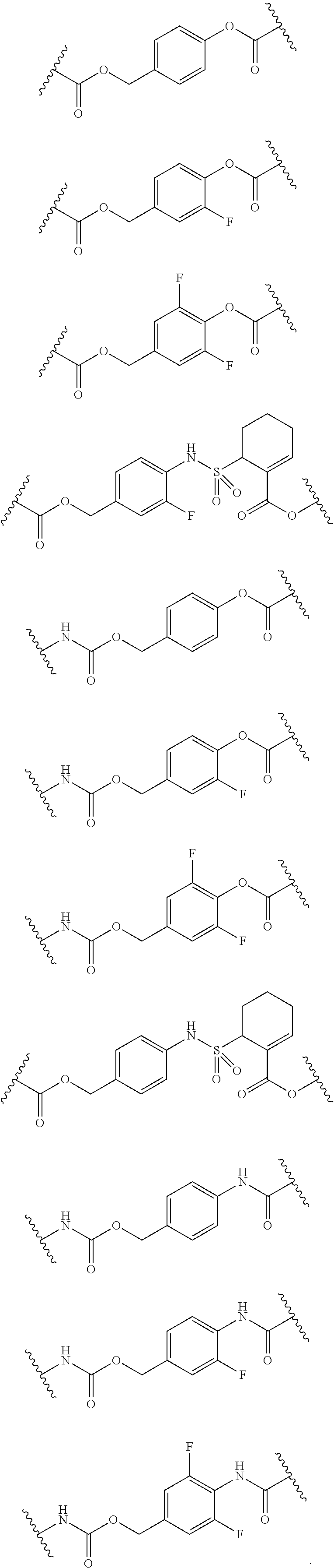

13. The composition of claim 11, wherein n is 2 at each occurrence.

14. The composition of claim 11, wherein the reactive group is or comprises —C(O)—O— or —O—C(O)—.

15. The composition of claim 11, wherein the reactive group comprises an aryl group, optionally bonded to —C(O)—O— or —O—C(O)— and substituted with one or more electron-withdrawing groups.

16. The composition of claim 11, wherein the aryl group has the structure of 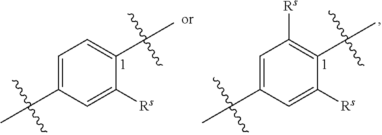
wherein R^{S} is independently chosen at each occurrence from halogen, —NO₂, —F, -L-R′, —C(O)-L-R′, —S(O)-L-R′, —S(O)₂-L-R′, and —P(O)(-L-R′)₂, and R′ is H or C₁-C₆ alkyl.

17. The composition of claim 2, wherein the first compound and the second compound are present in equimolar amount.

18. The composition of claim 2, wherein in formula (R-I):
the target agent is an antibody comprising an IgG heavy chain comprising K246 or K248, and
the target binding moiety is configured to bind the antibody so as to bring the reactive group in proximity with K246 or K248 of the IgG heavy chain to enable a reaction between K246 or K248 and the reactive group that results in attachment of a moiety comprising L^{RM}-MOI to K246 or K248 and expulsion of the group containing a target binding moiety from the compound.

19. A composition comprising:
a first compound having the structure of formula (P-II): 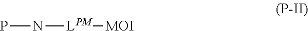
wherein:
P-N is a protein agent moiety comprising a lysine residue;
L^{PM} is a linker; and
MOI is a moiety of interest; and at least one of
a second compound having the structure: 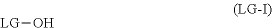
wherein LG is a group comprising a target binding moiety that binds to a target agent; and
a third compound having the formula (R-I): 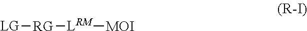
LG is a group comprising a target binding moiety that binds to a target agent, which is identical to LG in formula (LG-I);
RG is a reactive group;
L^{RM} is a linker, which is identical to in formula (P-II); and
MOI is a moiety of interest.

20. The composition of claim 19, further comprising:
a fourth compound having the formula (R-III): 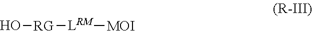
or a combination thereof.


SEQUENCE LISTING (3 sequences)

SEQ ID NO: 1 - protein, 13 aa, Artificial Sequence
  Synthetic
  di-sulfid (2)..(12)
DCAWXLGELVWCT

SEQ ID NO: 2 - protein, 56 aa, Artificial sequence
  Synthetic
DKEWILQKIYEIMRLLDELGHAEASMRVSDLIYEFMKKGDERLLEEAERLLEEVER

SEQ ID NO: 3 - protein, 64 aa, Artificial Sequence
  Synthetic
NDDELHMLMTDLVYEALHFAKDEEIKKRVFQLFELADKAYKNNDRQKLEKVVEELKELLERLLS